(19) United States
(12) Patent Application Publication
(10) Pub. No.: US 2023/0298707 A1
(43) Pub. Date: Sep. 21, 2023

(54) SYSTEMS AND METHODS FOR CLINICAL TRIAL RESULTS ENDPOINT-BASED ANALYSIS AND DYNAMIC AGGREGATION

(71) Applicant: Sumitomo Pharma Co., Ltd., Osaka (JP)

(72) Inventors: Julia Hyde GRAY, New York, NY (US); Mingzhe TAO, Fairfax, VA (US)

(73) Assignee: Sumitomo Pharma Co., Ltd., Osaka (JP)

(21) Appl. No.: 18/120,967

(22) Filed: Mar. 13, 2023

Related U.S. Application Data
(60) Provisional application No. 63/320,127, filed on Mar. 15, 2022.
(60) Provisional application No. 63/449,856, filed on Mar. 3, 2023.

(51) Int. Cl. G16H 10/20

(52) U.S. Cl. CPC G16H 10/20

(57) ABSTRACT

A computer-implemented method for clinical trial results endpoint-based analysis and dynamic aggregation, comprising the steps of receiving one or more selected clinical trials, wherein the one or more selected clinical trials match a specification; obtaining clinical trial results for the one or more selected clinical trials from at least one external data source; and interpreting, via a machine learning model, the obtained clinical trial results; importing the obtained clinical trial results as structured data. The method further comprising the steps of matching, based on a similarity analysis, via a processor, clinical trial endpoints identified in the obtained clinical trial results to corresponding normalized endpoint options; aggregating, based on the matched corresponding normalized endpoint options, the obtained clinical trial results to determine aggregated results; and providing the aggregated results.

### CROSS-REFERENCE TO RELATED APPLICATIONS

The present application claims the benefit of U.S. Patent Application No. 63/320,127 for CLINICAL TRIAL RESULTS AGGREGATION, filed Mar. 15, 2022, and U.S. Patent Application No. 63/449,856 for SYSTEMS AND METHODS FOR CLINICAL TRIAL RESULTS ENDPOINT-BASED ANALYSIS AND DYNAMIC AGGREGATION, filed Mar. 3, 2023, the entire contents of which are incorporated herein by reference in their entirety.

### FIELD OF THE INVENTION

The present invention is in the field of data analysis, specifically clinical trial results aggregation via endpoint analysis.

### INTRODUCTION

Data analysis is a process for converting information into a more useful form for supporting decision-making or drawing conclusions. Typical data analysis steps include collecting data, organizing data, manipulating data, and/or summarizing data. In many scenarios, a specific goal of data analysis is to select a collection of data items that are substantially similar to one another, in a specified and quantifiable sense, to one another. Alternatively, another goal of data analysis is to match data items or collections of data items based on other specified criteria. Accomplishing these goals may require complex and automated processing. This can be challenging, particularly in the context of data analysis of large amounts of data. Thus, it would be beneficial to develop techniques directed toward characterization of data for robust and efficient comparison.

Specifically, there is difficulty in analyzing clinical trial data. For example, clinical trial data analysis may be configured to determine whether such clinical trial results are relevant to a given disease. Considering there are swaths of clinical trial data related to a given disease, there is a great technical challenge in aggregating and, subsequently, analyzing such substantial collections of data. Further, a user may face difficulty in searching through such collections of data because of variations between a selected search term and variants thereof that are present in the data. For example, a user may fail to uncover conceptually relevant clinical trial results because the search term may not literally appear in said clinical trial results.

Accordingly, it would be desirable to provide systems and methods configured to aggregate and analyze clinical trial data. Yet further, it would be desirable to provide systems and method configured to automatically identify clinical trial results that are relevant to a particular disease indication in a computationally practicable manner.

### SUMMARY

In accordance with the present disclosure, the following items are provided.

(Item 1). A computer-implemented method, comprising the steps of:
receiving a one or more selected clinical trials,
wherein the one or more selected clinical trials match a specification;
obtaining a set of clinical trial results for the one or more selected clinical trials from a at least one external data source;
interpreting, via a machine learning model, the set of clinical trial results;
importing the set of clinical trial results in a structured data format;
matching, based on a similarity analysis, via a processor, a set of clinical trial endpoints identified in the set of clinical trial results to a set of corresponding normalized endpoint options;
aggregating, based on the set of corresponding normalized endpoint options, the set of clinical trial results to determine a set of aggregated results; and providing the set of aggregated results.

(Item 2). The computer-implemented method of Item 1, wherein the one or more selected clinical trials are provided from a list comprising a one or more clinical trials matching the specification.

(Item 3). The computer-implemented method of Item 2, wherein the list identifies which of the one or more clinical trials matching the specification include a set of clinical result data obtainable from the at least one external data source.

(Item 4). The computer-implemented method of any one of Items 1 to 3, wherein the specification comprises a disease category.

(Item 5). The computer-implemented method of Item 4, wherein the specification further comprises a specific disease within the disease category.

(Item 6). The computer-implemented method of any one of Items 1 to 5, wherein the specification comprises a clinical trial phase category.

(Item 7). The computer-implemented method of any one of Items 1 to 6, further comprising the step of receiving, from a user, the specification via a graphical user interface.

(Item 8). The computer-implemented method of any one of Items 1 to 7, wherein the at least one external data source comprises an online database of clinical trial data maintained by an at least one government entity responsible for regulating clinical trials, international agencies, university network organizations, organizations of medical associations, or foundations based on an association of pharmaceutical manufacturers.

(Item 9). The computer-implemented method of any one of Items 1 to 8, wherein the machine learning model includes a named entity recognition (NER) model.

(Item 10). The computer-implemented method of Item 9, wherein the NER model utilizes a recurrent neural network (RNN) architecture.

(Item 11). The computer-implemented method of any one of Items 1 to 10, wherein interpreting, via the machine learning model, the set of clinical trial results further comprises automatically extracting specified text from unstructured text of the set of clinical trial results.

(Item 12). The computer-implemented method of any one of Items 1 to 11, wherein the structured data format comprises a collection of fields corresponding to categories of syntactic units extracted from the set of clinical trial results.

(Item 13). The computer-implemented method of any one of Items 1 to 12, wherein the similarity analysis comprises at least a computation of a word distance metric between the set of clinical trial endpoints identified in the set of clinical trial results and the set of corresponding normalized endpoint options.

(Item 14). The computer-implemented method of any one of Items 1 to 13, further comprising the steps of:
generating a one or more confirmation selection tools, the one or more confirmation selection tools corresponding to the set of corresponding normalized endpoint options; and receiving, from a user, actuation of one or more of the one or more confirmation selection tools.

(Item 15). The computer-implemented method of any one of Items 1 to 14, wherein the set of aggregated results are ordered based on a match score of each result of the set of aggregated results.

(Item 16). The computer-implemented method of any one of Items 1 to 15, wherein the set of aggregated results comprise a tabular structure comprising a one or more columns and a one or more rows, and wherein the one or more columns represent different clinical trials and the one or more rows represent different clinical trial properties.

(Item 17). The computer-implemented method of any one of Items 1 to 16, further comprising one or more selected from the group comprised of: filtering, machine translating, and standardizing terminology of the one or more selected clinical trials before obtaining the set of clinical trial results from the at least the one external data source.

(Item 18). The computer-implemented method of any one of Items 1 to 17, wherein the machine learning model has been trained on a set of training datasets comprising a constrained set of collections of text with prescribed clinical endpoint categories to which the set of clinical trial endpoints identified in the set of clinical trial results belong.

(Item 19). A system, comprising:
a server comprising at least one server processor, at least one server database, at least one server memory comprising a set of computer-executable server instructions which, when executed by the at least one server processor, cause the server to:
receive a one or more selected clinical trials,
wherein the one or more selected clinical trials match a specification;
obtain a set of clinical trial results for the one or more selected clinical trials from a at least one external data source;
interpret, via a machine learning model, the set of clinical trial results;
import the set of clinical trial results in a structured data format;
match, based on a similarity analysis, via a processor, a set of clinical trial endpoints identified in the set of clinical trial results to a set of corresponding normalized endpoint options;
aggregate, based on the set of corresponding normalized endpoint options, the set of clinical trial results to determine a set of aggregated results; and
provide the set of aggregated results.

(Item 20). The system of Item 19, wherein the one or more selected clinical trials are provided from a list comprising a one or more clinical trials matching the specification.

(Item 21). The system of Item 20, wherein the list identifies which of the one or more clinical trials matching the specification include a set of clinical result data obtainable from the at least one external data source.

(Item 22). The system of any one of Items 19 to 21, wherein the specification comprises a disease category.

(Item 23). The system of Item 22, wherein the specification further comprises a specific disease within the disease category.

(Item 24). The system of any one of Items 19 to 23, wherein the specification comprises a clinical trial phase category.

(Item 25). The system of any one of Items 19 to 24, further comprising a client device comprising at least one device processor, at least one display, at least one device memory comprising a set of computer-executable device instructions which, when executed by the at least one device processor, cause the client device to receive, from a user, the specification via a graphical user interface.

(Item 26). The system of any one of Items 19 to 25, wherein the at least one external data source comprises an online database of clinical trial data maintained by an at least one government entity responsible for regulating clinical trials, international agencies, university network organizations, organizations of medical associations, or foundations based on an association of pharmaceutical manufacturers.

(Item 27). The system of any one of Items 19 to 26, wherein the machine learning model includes a named entity recognition (NER) model.

(Item 28). The system of Item 27, wherein the NER model utilizes a recurrent neural network (RNN) architecture.

(Item 29). The system of any one of Items 19 to 28, wherein the set of computer-executable server instructions which, when executed by the at least one server processor, cause the server to interpret, via the machine learning model, the set of clinical trial results further cause the server to automatically extract specified text from unstructured text of the set of clinical trial results.

(Item 30). The system of any one of Items 19 to 29, wherein the structured data format comprises a collection of fields corresponding to categories of syntactic units extracted from the set of clinical trial results.

(Item 31). The system of any one of Items 19 to 30, wherein the similarity analysis comprises at least a computation of a word distance metric between the set of clinical trial endpoints identified in the set of clinical trial results and the set of corresponding normalized endpoint options.

(Item 32). The system of any one of Items 19 to 31, wherein the set of computer-executable server instructions which, when executed by the at least one server processor, further cause the server to:
generate a one or more confirmation selection tools, the one or more confirmation selection tools corresponding to the set of corresponding normalized endpoint options; and
receive, from a user, actuation of one or more of the one or more confirmation selection tools.

(Item 33). The system of any one of Items 19 to 32, wherein the set of aggregated results are ordered based on a match score of each result of the set of aggregated results.

(Item 34). The system of any one of Items 19 to 33, wherein the set of aggregated results comprise a tabular structure comprising a one or more columns and a one or more rows, and wherein the one or more columns represent different clinical trials and the one or more rows represent different clinical trial properties.

(Item 35). The system of any one of Items 19 to 34, wherein the set of computer-executable server instructions which, when executed by the at least one server processor, further cause the server to execute one or more selected from the group comprised of: filter, machine translate, and standardize terminology of the one or more selected clinical trials before obtaining the set of clinical trial results from the at least the one external data source.

(Item 36). The system of any one of Items 19 to 35, wherein the machine learning model has been trained on a set of training datasets comprising a constrained set of collections of text with prescribed clinical endpoint categories to which the set of clinical trial endpoints identified in the set of clinical trial results belong.

(Item 37). A non-transitory computer readable medium having a set of instructions stored thereon that, when executed by a processing device, cause the processing device to carry out an operation of clinical result aggregation, the operation comprising:
receiving a one or more selected clinical trials,
wherein the one or more selected clinical trials match a specification;
obtaining a set of clinical trial results for the one or more selected clinical trials from a at least one external data source;
interpreting, via a machine learning model, the set of clinical trial results;
importing the set of clinical trial results in a structured data format;
matching, based on a similarity analysis, via a processor, a set of clinical trial endpoints identified in the set of clinical trial results to a set of corresponding normalized endpoint options;
aggregating, based on the set of corresponding normalized endpoint options, the set of clinical trial results to determine a set of aggregated results; and
providing the set of aggregated results.

(Item 38). The non-transitory computer readable medium of Item 37, wherein the one or more selected clinical trials are provided from a list comprising a one or more clinical trials matching the specification.

(Item 39). The non-transitory computer readable medium of Item 38, wherein the list identifies which of the one or more clinical trials matching the specification include a set of clinical result data obtainable from the at least one external data source.

(Item 40). The non-transitory computer readable medium of any one of Items 37 to 39, wherein the specification comprises a disease category.

(Item 41). The non-transitory computer readable medium of Item 40, wherein the specification further comprises a specific disease within the disease category.

(Item 42). The non-transitory computer readable medium of any one of Items 37 to 41, wherein the specification comprises a clinical trial phase category.

(Item 43). The non-transitory computer readable medium of any one of Items 37 to 42, the operation comprising further comprising receiving, from a user, the specification via a graphical user interface.

(Item 44). The non-transitory computer readable medium of any one of Items 37 to 43, wherein the at least one external data source comprises an online database of clinical trial data maintained by an at least one government entity responsible for regulating clinical trials, international agencies, university network organizations, organizations of medical associations, or foundations based on an association of pharmaceutical manufacturers.

(Item 45). The non-transitory computer readable medium of any one of Items 37 to 44, wherein the machine learning model includes a named entity recognition (NER) model.

(Item 46). The non-transitory computer readable medium of Item 45, wherein the NER model utilizes a recurrent neural network (RNN) architecture.

(Item 47). The non-transitory computer readable medium of any one of Items 37 to 46, wherein interpreting, via the machine learning model, the set of clinical trial results further comprises automatically extracting specified text from unstructured text of the set of clinical trial results.

(Item 48). The non-transitory computer readable medium of any one of Items 37 to 47, wherein the structured data format comprises a collection of fields corresponding to categories of syntactic units extracted from the set of clinical trial results.

(Item 49). The non-transitory computer readable medium of any one of Items 37 to 48, wherein the similarity analysis comprises at least a computation of a word distance metric between the set of clinical trial endpoints identified in the set of clinical trial results and the set of corresponding normalized endpoint options.

(Item 50). The non-transitory computer readable medium of any one of Items 37 to 49, the operation further comprising:
generating a one or more confirmation selection tools, the one or more confirmation selection tools corresponding to the set of corresponding normalized endpoint options; and receiving, from a user, actuation of one or more of the one or more confirmation selection tools.

(Item 51). The non-transitory computer readable medium of any one of Items 37 to 50, wherein the set of aggregated results are ordered based on a match score of each result of the set of aggregated results.

(Item 52). The non-transitory computer readable medium of any one of Items 37 to 51, wherein the set of aggregated results comprise a tabular structure comprising a one or more columns and a one or more rows, and wherein the one or more columns represent different clinical trials and the one or more rows represent different clinical trial properties.

(Item 53). The non-transitory computer readable medium of any one of Items 37 to 52, the operation further comprising one or more selected from the group comprised of: filtering, machine translating, and standardizing terminology of the one or more selected clinical trials before obtaining the set of clinical trial results from the at least the one external data source.

(Item 54). The non-transitory computer readable medium of any one of Items 37 to 53, wherein the machine learning model has been trained on a set of training datasets comprising a constrained set of collections of text with prescribed clinical endpoint categories to which the set of clinical trial endpoints identified in the set of clinical trial results belong.

(Item 55). A computer-implemented method, comprising the steps of:
receiving, from a user, the specification via a graphical user interface;
receiving a one or more selected clinical trials,
wherein the one or more selected clinical trials match a specification,
wherein the one or more selected clinical trials are provided from a list comprising a one or more clinical trials matching the specification, and
wherein the list identifies which of the one or more clinical trials matching the specification include a set of clinical result data obtainable from the at least one external data source;
obtaining a set of clinical trial results for the one or more selected clinical trials from a at least one external data source;
interpreting, via a machine learning model, the set of clinical trial results;
importing the set of clinical trial results in a structured data format;
matching, based on a similarity analysis, via a processor, a set of clinical trial endpoints identified in the set of clinical trial results to a set of corresponding normalized endpoint options;
aggregating, based on the set of corresponding normalized endpoint options, the set of clinical trial results to determine a set of aggregated results;
providing the set of aggregated results;
generating a one or more confirmation selection tools, the one or more confirmation selection tools corresponding to the set of corresponding normalized endpoint options; and
receiving, from a user, actuation of one or more of the one or more confirmation selection tools.

(Item 56). A system, comprising:
a server comprising at least one server processor, at least one server database, at least one server memory comprising a set of computer-executable server instructions which, when executed by the at least one server processor, cause the server to:
receive a one or more selected clinical trials,
wherein the one or more selected clinical trials match a specification;
obtain a set of clinical trial results for the one or more selected clinical trials from a at least one external data source;
interpret, via a machine learning model, the set of clinical trial results and automatically extract specified text from unstructured text of the set of clinical trial results,
wherein the machine learning model includes a named entity recognition (NER) model, and
wherein the NER model utilizes a recurrent neural network (RNN) architecture;
import the set of clinical trial results in a structured data format;
match, based on a similarity analysis, via a processor, a set of clinical trial endpoints identified in the set of clinical trial results to a set of corresponding normalized endpoint options,
wherein the similarity analysis comprises at least a computation of a word distance metric between the set of clinical trial endpoints identified in the set of clinical trial results and the set of corresponding normalized endpoint options;
aggregate, based on the set of corresponding normalized endpoint options, the set of clinical trial results to determine a set of aggregated results; and
provide the set of aggregated results.

(Item 57). A non-transitory computer readable medium having a set of instructions stored thereon that, when executed by a processing device, cause the processing device to carry out an operation of clinical result aggregation, the operation comprising:
receiving a one or more selected clinical trials,
wherein the one or more selected clinical trials match a specification;
obtaining a set of clinical trial results for the one or more selected clinical trials from a at least one external data source;
interpreting, via a machine learning model, the set of clinical trial results,
wherein the machine learning model has been trained on a set of training datasets comprising a constrained set of collections of text with prescribed clinical endpoint categories to which the set of clinical trial endpoints identified in the set of clinical trial results belong;
importing the set of clinical trial results in a structured data format,
wherein the structured data format comprises a collection of fields corresponding to categories of syntactic units extracted from the set of clinical trial results;
matching, based on a similarity analysis, via a processor, a set of clinical trial endpoints identified in the set of clinical trial results to a set of corresponding normalized endpoint options;
aggregating, based on the set of corresponding normalized endpoint options, the set of clinical trial results to determine a set of aggregated results,
wherein the set of aggregated results are ordered based on a match score of each result of the set of aggregated results,
wherein the set of aggregated results comprise a tabular structure comprising a one or more columns and a one or more rows, and
wherein the one or more columns represent different clinical trials and the one or more rows represent different clinical trial properties; and
providing the set of aggregated results.

(Item 58). A computer-implemented method, comprising the steps of:
receiving a one or more selected clinical trials;
obtaining a set of clinical trial results for the one or more selected clinical trials;
interpreting, via a machine learning model, the set of clinical trial results;
matching, based on a similarity analysis, via a processor, a set of clinical trial endpoints identified in the set of clinical trial results to a set of corresponding normalized endpoint options; and
aggregating, based on the set of corresponding normalized endpoint options, the set of clinical trial results to determine a set of aggregated results.

Additional aspects related to this disclosure are set forth, in part, in the description which follows, and, in part, will be obvious from the description, or may be learned by practice of this disclosure.

It is to be understood that both the foregoing and the following descriptions are exemplary and explanatory only and are not intended to limit the claimed disclosure or application thereof in any manner whatsoever.

### BRIEF DESCRIPTION OF THE DRAWINGS

The incorporated drawings, which are incorporated in and constitute a part of this specification exemplify the aspects of the present disclosure and, together with the description, explain and illustrate principles of this disclosure.

FIG. 1 is a block diagram illustrating an embodiment of a system for obtaining and aggregating clinical trial results.

FIG. 2 is a flow diagram illustrating an embodiment of a process for obtaining and aggregating clinical trial results.

FIG. 3 is a flow diagram illustrating an embodiment of a process for identifying clinical trials matching a specification.

FIG. 4 is a flow diagram illustrating an embodiment of a process for importing clinical trial results as structured data.

FIG. 5 is a flow diagram illustrating an embodiment of a process for determining aggregated results.

FIGS. 6A-6I illustrate various user interface elements of a system for obtaining and aggregating clinical trial results.

FIG. 7 is a functional diagram illustrating a programmed computer system that can implement one or more aspects of an embodiment of the invention.

FIG. 8 illustrates a block diagram of a distributed computer system that can implement one or more aspects of an embodiment of the present invention.

### DETAILED DESCRIPTION

In the following detailed description, reference will be made to the accompanying drawing(s), in which identical functional elements are designated with like numerals. The aforementioned accompanying drawings show by way of illustration, and not by way of limitation, specific aspects, and implementations consistent with principles of this disclosure. These implementations are described in sufficient detail to enable those skilled in the art to practice the disclosure and it is to be understood that other implementations may be utilized and that structural changes and/or substitutions of various elements may be made without departing from the scope and spirit of this disclosure. The following detailed description is, therefore, not to be construed in a limited sense.

It is noted that description herein is not intended as an extensive overview, and as such, concepts may be simplified in the interests of clarity and brevity. As used herein, a “set” may refer generally to one or more of the item to which it relates. Thus, items appended with the language of “set” or “one or more” may be interpreted as one or more of the item.

All documents mentioned in this application are hereby incorporated by reference in their entirety. Any process described in this application may be performed in any order and may omit any of the steps in the process. Processes may also be combined with other processes or steps of other processes.

The present disclosure relates to systems and methods for clinical trial results aggregation.

The invention of the present disclosure may be a system and/or method configured for clinical trial results aggregation. Thus, an identification of one or more selected clinical trials among one or more clinical trials matching a specification may be received. Clinical trial results for the selected clinical trials may be obtained from at least one external data source. A language model based on machine learning techniques may be used to interpret the obtained clinical trial results and import the obtained clinical trial results as structured data. In an embodiment, based on a similarity analysis, clinical trial endpoints identified in the obtained clinical trial results are matched to corresponding normalized endpoint options. The matched corresponding normalized endpoint options may be used to aggregate the obtained clinical trial results to determine aggregated results.

The techniques disclosed herein address the technical problem of automatically determining a collection of clinical trial results that are relevant to a particular disease indication in a computationally practicable manner. Automatically collecting clinical trial results for the particular disease indication is technically challenging because there are multiple sources for data related to clinical trials and these sources have large amounts of data, making it computationally difficult to determine the desired data in a computationally practicable time frame. For example, it is often the case that a primary source of clinical trial outcome data is not configured for a flexible search (e.g., by disease indication or other parameters). Thus, a solution is to first search another data source that does not contain all the data as the primary source but is associated with the primary source and is more amenable to a comprehensive search. Various technical challenges are associated with doing this, and these technical challenges are addressed by the techniques disclosed herein. For example, words intended to be surfaced by the first search may have numerous potential lexicological variants, making them difficult to locate and compare with standardized terms. As described herein, technical solutions to address this variation issue include utilizing a machine learning model for extraction and utilizing distance metrics for comparison. In some embodiments, a preprocessing process that includes machine translation (e.g., translation to English in situations in which an original database of clinical trial results is in a non-English language), standardization of terminology, and/or other processing is performed.

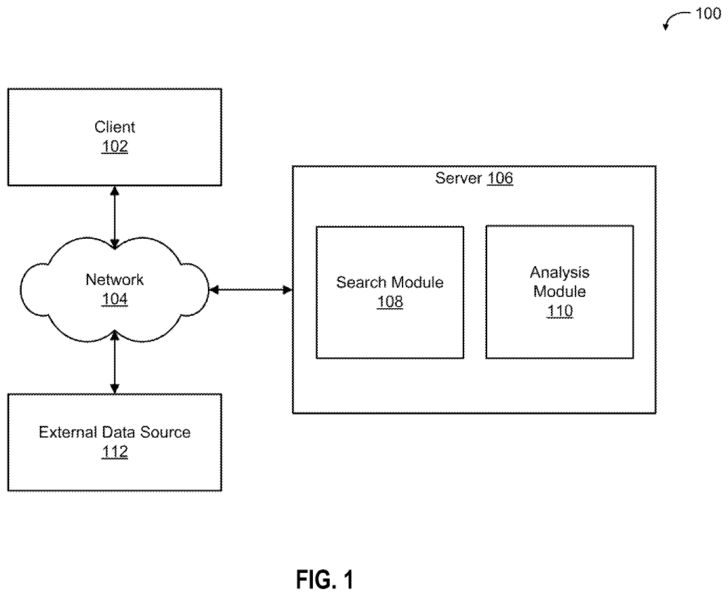

FIG. 1 is a block diagram illustrating an embodiment of a system for obtaining and aggregating clinical trial results. In the example shown, system 100 includes client device 102, network 104, server 106, and external data source 112. Server 106, in the example illustrated, includes search module 108 and analysis module 110. The number of components and the connections shown in FIG. 1 are merely illustrative. Other system architectures that implement the techniques disclosed herein are also possible.

In various embodiments, client device 102 is a computer or other hardware device that a user utilizes to request data and/or view responses. Examples of client hardware devices include desktop computers, laptop computers, tablets, smartphones, virtual reality (VR) headsets, augmented reality (AR) glasses, and other devices. In various embodiments, the client hardware device includes a software user interface through which the user can perform data access and other user interface operations. For example, the software user interface may be a web portal, internal network portal, or other portal that allows the user to submit queries and graphically view and interact with received results. Other examples of software include browsers, mobile apps, chat clients, etc.

In the example illustrated, client device 102, server 106, and external data source 112 are communicatively connected via network 104. Requests may be transmitted to and responses received from server 106 using network 104. Examples of network 104 include one or more of the following: a direct or indirect physical communication connection, mobile communication network, Internet, intranet, Local Area Network, Wide Area Network, Storage Area Network, and any other form of connecting two or more systems, components, or storage devices together.

In various embodiments, server 106 is a computer or other hardware component that provides clinical trial data search and analysis functionality. In the example illustrated, search module 108 and analysis module 110 reside on server 106. In various embodiments, search module 108 and analysis module 110 are computer software aspects. In various embodiments, server 106 comprises software configured to aggregate clinical trial data in a manner such that the clinical trial data can be compared systematically. For example, clinical trials with the same endpoints (e.g., a same primary endpoint or same secondary endpoint) can be aggregated. As used herein, an endpoint (also referred to as a clinical endpoint, clinical trial endpoint, and so forth) refers to an event or outcome that can be measured objectively to determine whether an intervention being studied is beneficial, whether a clinical trial associated with the intervention should end, or other characteristics related to clinical trial events or outcomes. Examples of endpoints are survival, improvements in quality of life, and relief of symptoms. In various embodiments, using the techniques disclosed herein, endpoints are normalized so said endpoints can be compared across different clinical trials.

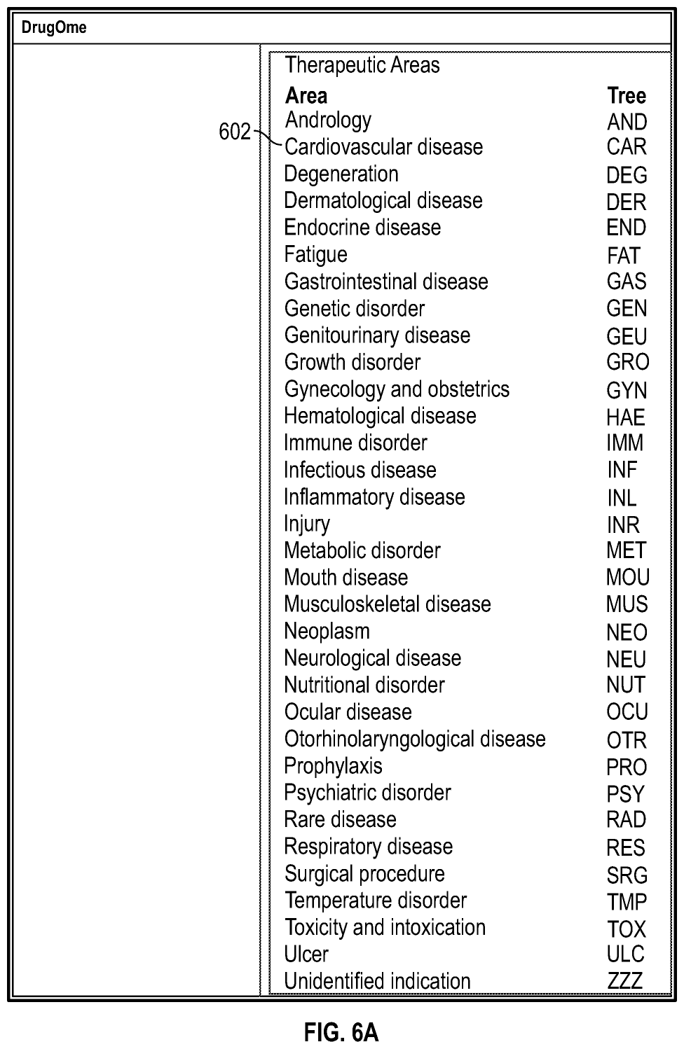

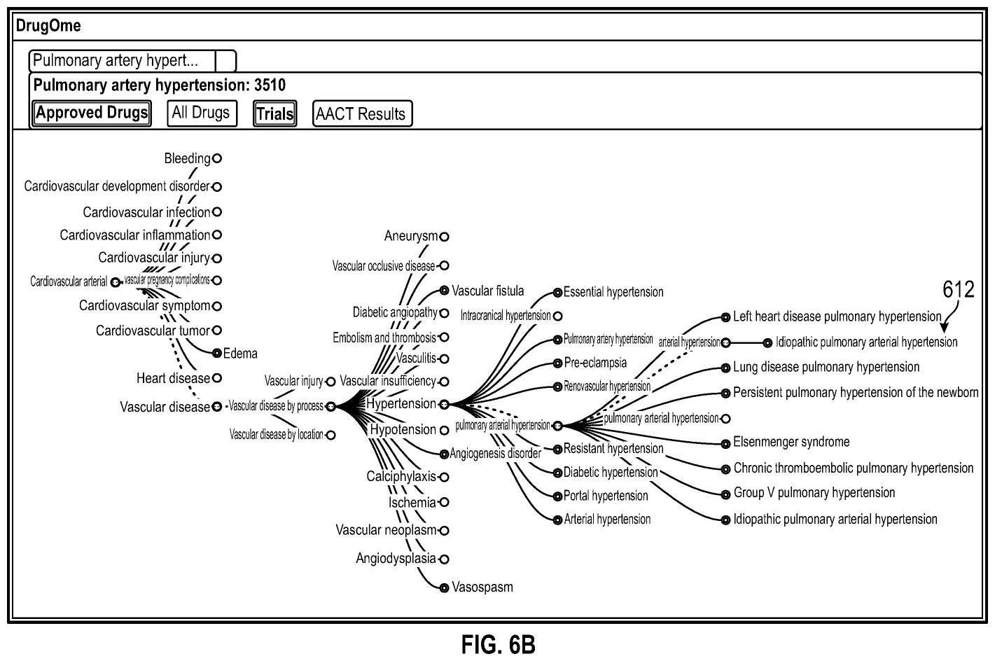

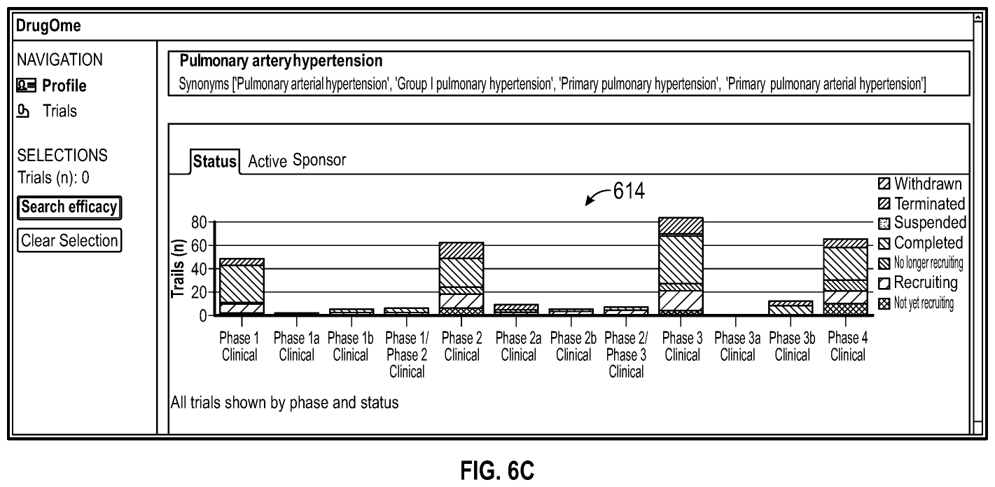

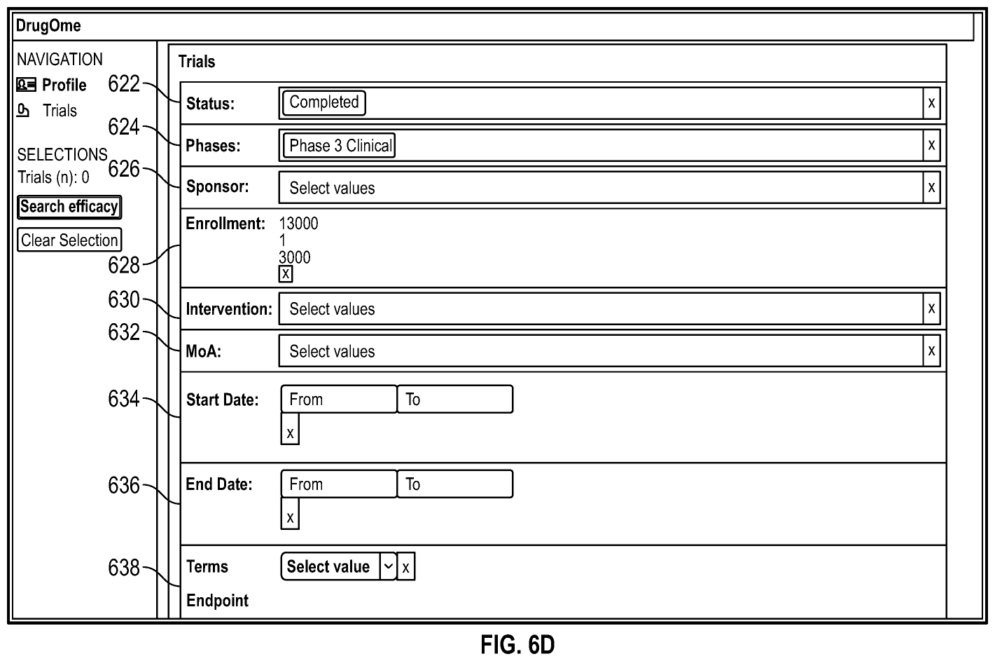

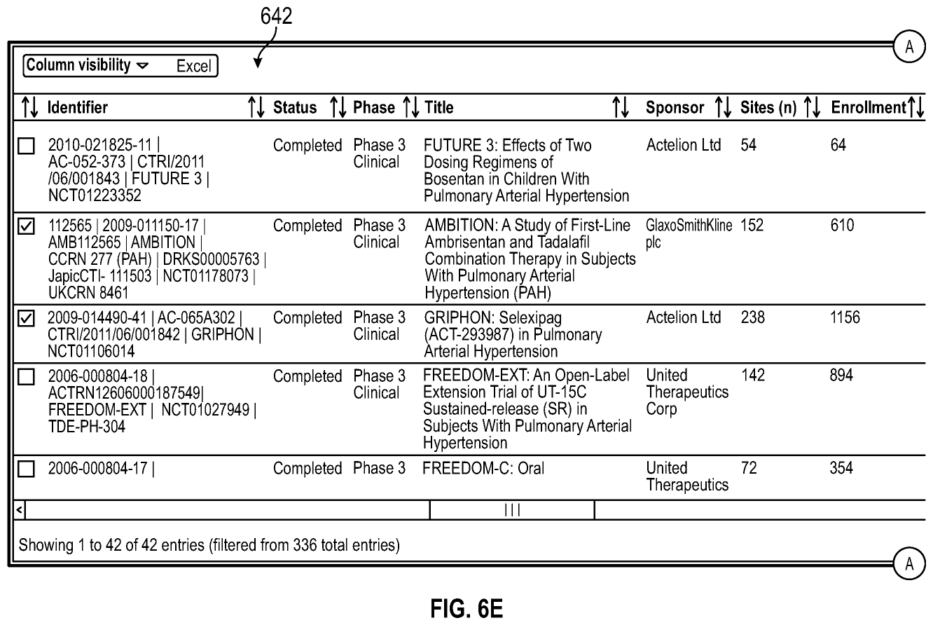

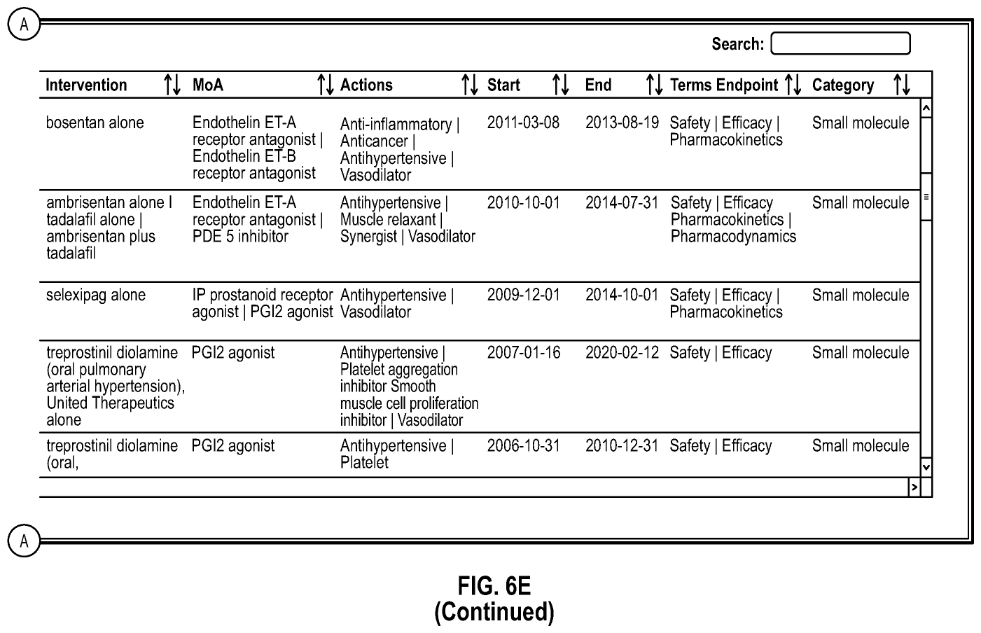

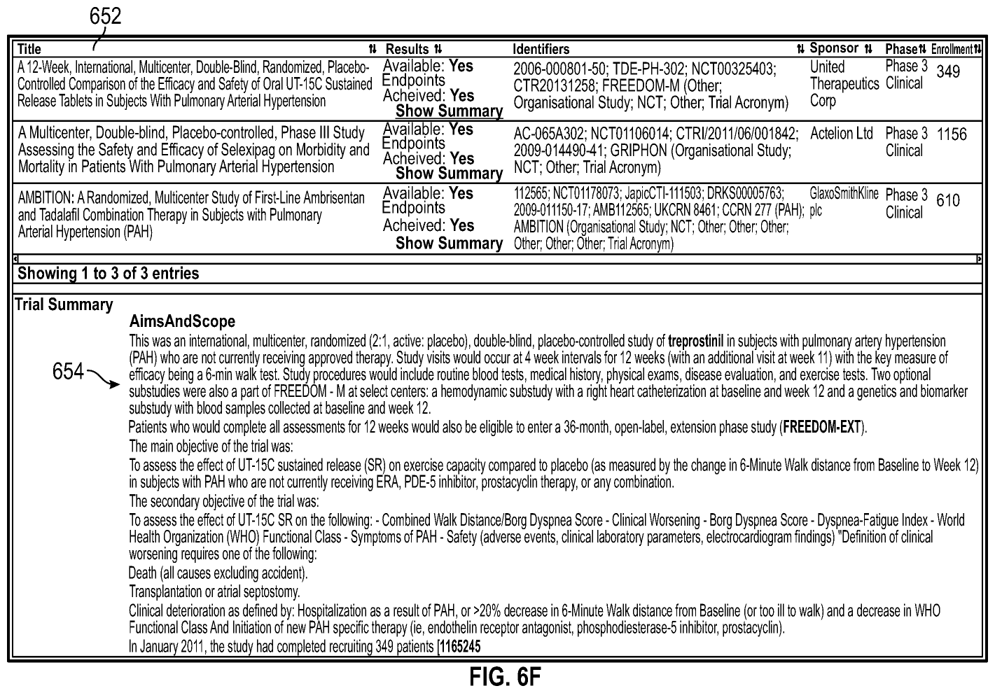

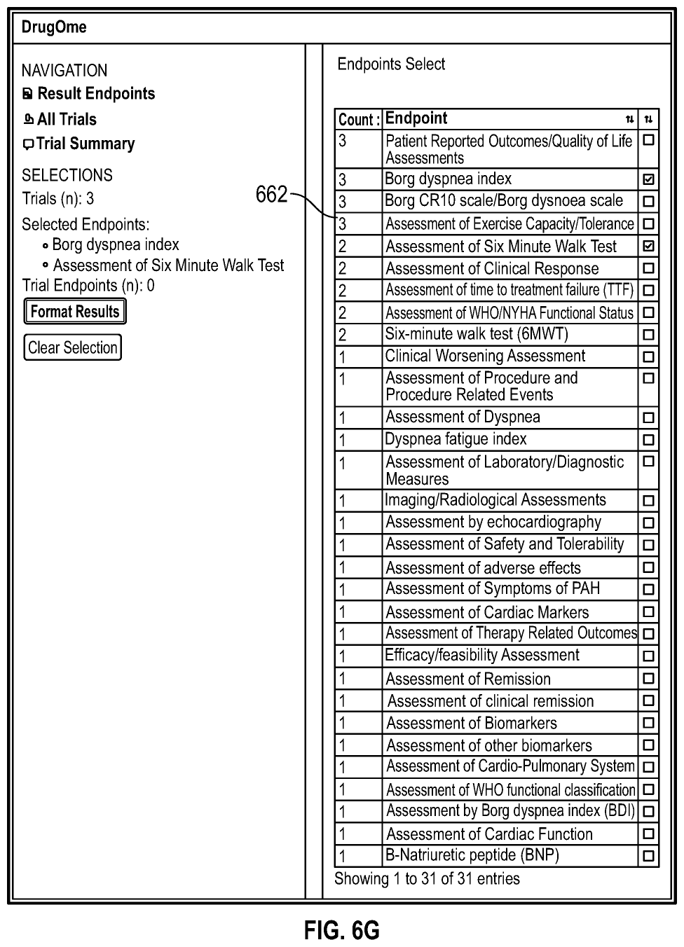

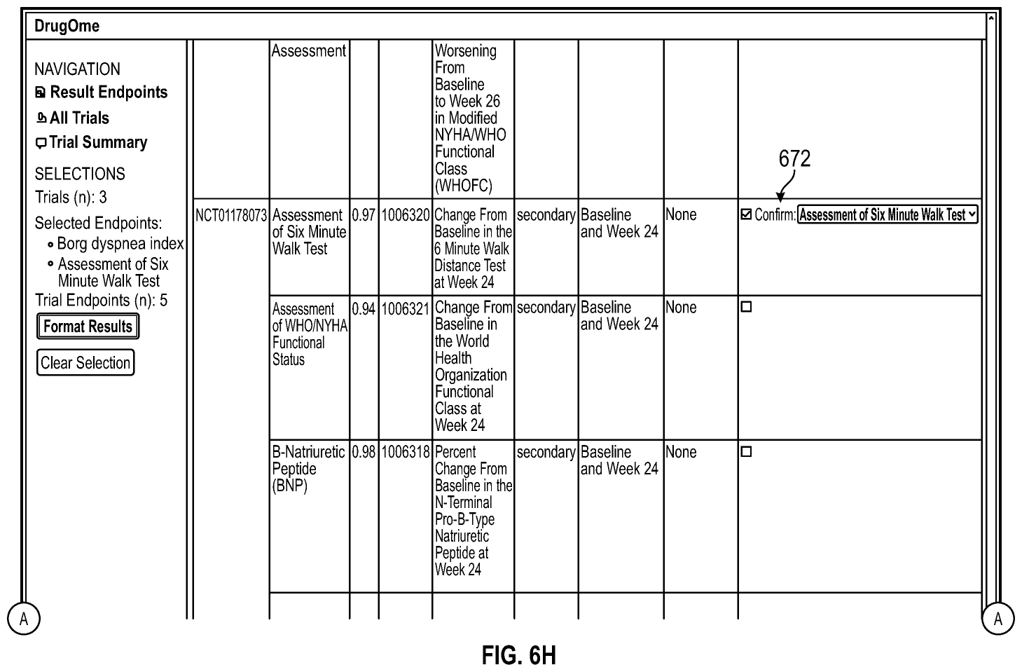

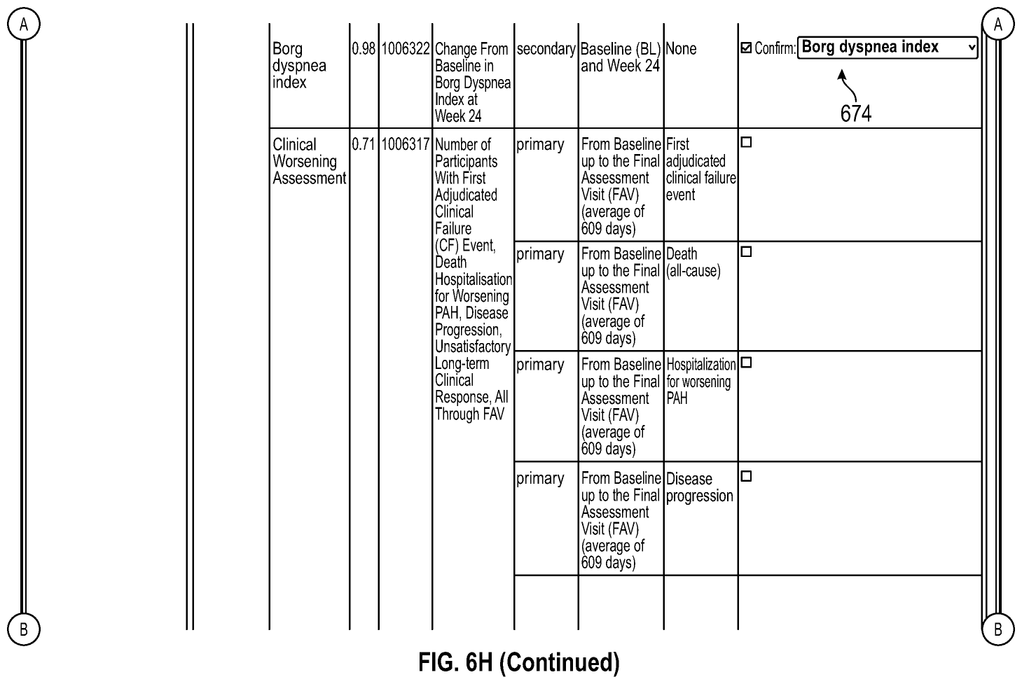

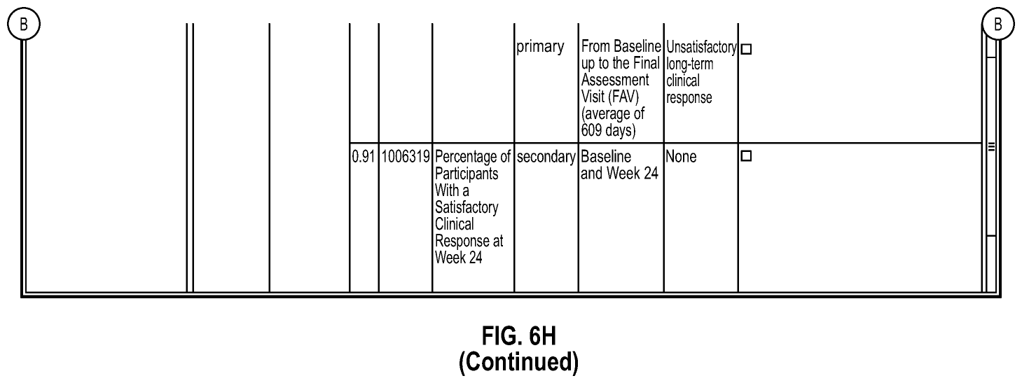

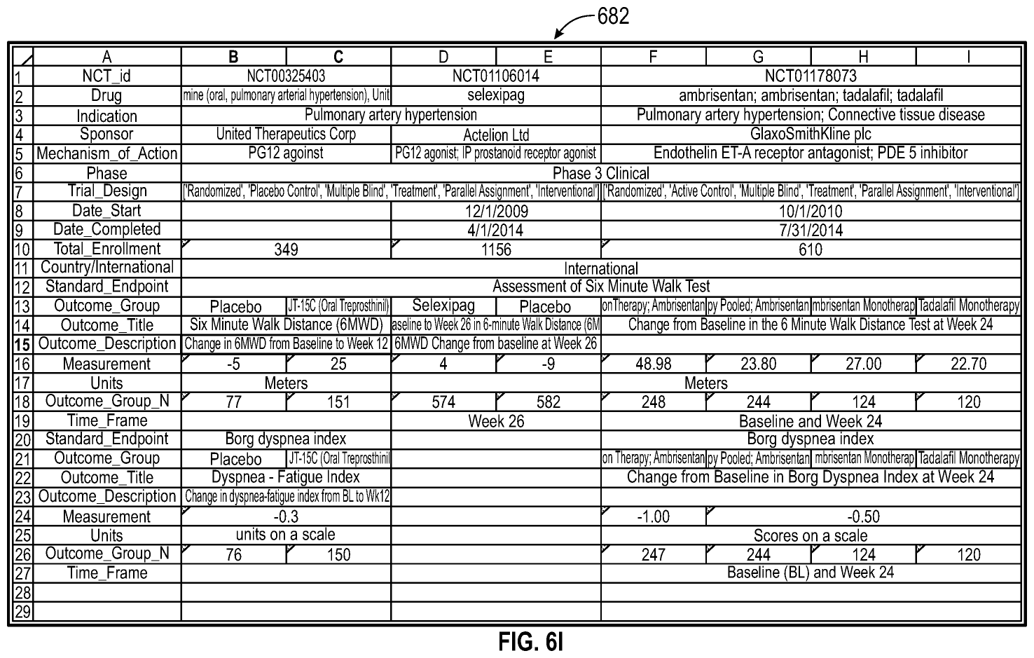

In various embodiments, search module 108 is configured to identify clinical trials matching specified criteria or text. In some embodiments, the specified criteria are received by search module 108 as an input from client device 102. For example, the input may include a therapeutic area that is targeted by the clinical trials to be identified. FIG. 6A illustrates an example of a user interface that may be presented to a user of client device 102 in which the user is prompted to select a therapeutic area from a list of therapeutic areas. While the area interface of FIG. 6A contemplates therapeutic areas, the area interface may be populated with any suitable categories or families. Various therapeutic areas are shown in the example of FIG. 6A, including therapeutic area 602, which is “cardiovascular disease” in the example illustrated. In some embodiments, the user is further prompted to select a sub-area associated with the selected therapeutic area. Accordingly, in instances of complex or large areas, the system may be configured to divide such an area into sub-areas. Said sub-areas may be populated on the area interface or a similar interface, wherein a user may select a desired sub-area. FIG. 6B illustrates an example of a user interface that may be presented to the user of client device 102 for selection of the sub-area. In an embodiment, the tree interface, as shown in FIG. 6B, a complete ontology tree may be generated and displayed. However, in another embodiment, branches or levels of the tree may generate and display based on the user's selection. As a non-limiting example, each item in a particular level of the tree may be selectable, wherein selection of an item generates and displays the next level of the tree if a succeeding level exists. In the example illustrated, the user has already selected cardiovascular disease as the therapeutic area, and various cardiovascular conditions can be selected using an ontology tree. In the example shown, the user has navigated through various branches of the ontology tree to arrive at disease indication selection 612, which is pulmonary arterial hypertension in the example illustrated. The disease indication selection process shown in FIGS. 6A and 6B is illustrative and not restrictive. Different selection processes (e.g., based on text search) may also be used.

In various embodiments, search module 108 receives additional criteria from a user of client device 102 to further narrow the clinical trials to be identified. As non-limiting examples, clinical trials can be narrowed according to clinical trial phase categories, e.g., according to whether clinical trials are phase 1, phase 1a, phase 1b, phase 2, phase 2a, phase 2b, phase 3, phase 3a, phase 3b, phase 4, etc. and according to clinical trial status, e.g., whether trials are ongoing, suspended, completed, or another status. FIG. 6C is an example of a visualization that may be reported to the user indicating a distribution of clinical trials associated with pulmonary arterial hypertension. Shown in results 614 are counts of various statuses (e.g., terminated, completed, and other statuses) associated with various phases of clinical trials from phase 1 to phase 4. In some embodiments, the user is able to select (e.g., by clicking on) a type of clinical trial (e.g., completed phase 3) as a criterion for narrowing clinical trial results to be presented to the user. It is also possible for the user to specify narrowing criteria via a filter menu. An example of a filter user interface is shown in FIG. 6D. For example, clinical trial status and clinical trial phase category can be selected via fields 622 and 624, respectively. In the example shown, these fields indicate the user has selected completed phase 3 clinical trials. Various other interactive filters may also be presented. As non-limiting examples, clinical trials can also be narrowed according to sponsor (e.g., an entity managing the clinical trial), enrollment number (e.g., number of participants in the clinical trial), intervention (e.g., medical product, drug, device, medical procedure, etc.), mechanism of action (MoA) (e.g., including a specific molecular target, such as an enzyme or receptor), start date (e.g., from a range), end date (e.g., from a range), and terms endpoint (e.g., whether the trial was measuring efficacy, safety, or was observational), which correspond to fields 626, 628, 630, 632, 634, 636, and 638, respectively. Accordingly, the filter interface may include a status field 622, a clinical trial phase category field 624, a sponsor field 626, an enrollment field 628, an intervention field 630, a MoA field 632, a start date field 634, an end date field 636, and/or a terms endpoint field 638, wherein any of the aforementioned fields may include a text box, drop-down menu, slider, or other means of selection making. The specific example of filtering by endpoint is described in further detail herein. In some embodiments, endpoints are refined and/or finalized after an initial list of clinical trials are obtained. Thus, the initial endpoint filtering may be presented at a high level (e.g., broadly related to safety, efficacy, etc.).

In various embodiments, filtered results can be viewed in a list format in which various properties of the identified clinical trials are also presented. Examples of these properties include a clinical trial identifier, status, phase, title, sponsor, number of sites, enrollment count, intervention, MoA, actions, start date, end date, endpoints, drug category, etc. An example of a display of filtered results in a user interface is shown in FIG. 6E, which shows a list of clinical trials 642. In an embodiment, an item from the list of clinical trials 642 can be selected, wherein such a selection generates and/or displays a summary for the item. For example, FIG. 6F shows a list of selected clinical trials 652, from which an item is selected to enable appearance of clinical trial summary 654 as a user interface element. In various embodiments, the user is able to finalize a list of clinical trials for which clinical trial result data is to be obtained. For example, the user may click on items from clinical trials list 642 and/or 652 to select clinical trials. These are considered the selected clinical trials that match the specified criteria (e.g., filters) for which clinical trial results are desired.

In various embodiments, search module 108 is configured to obtain clinical trial results for the selected clinical trials from at least one external data source. In some embodiments, server 106 obtains the clinical trial results from external data source 112 via network 104. In various embodiments, external data source 112 stores digital content items that comprise clinical trial results. Examples of digital content items include text-based documents (e.g., scientific articles or publications, press releases, news articles, books, websites converted into documents, and any other types of documents), images, audio files, video files, tabular files, slide presentation files, and any other types of content items that can be represented digitally. In some embodiments, external data source 112 spans multiple data sources (e.g., multiple Internet sources providing documents). In various embodiments, external data source 112 is a structured set of data held in one or more computers and/or storage devices. Examples of storage devices include hard disk drives and solid-state drives. In some embodiments, external data source 112 includes an online database of clinical trial data maintained by one or more government entities responsible for regulating clinical trials. It is also possible for clinical trial data to be maintained by other types of organizations, such as one or more international agencies, public agencies such as university network organizations, organizations of medical associations, foundations based on an association of pharmaceutical manufacturers, etc. As a specific example, with respect to the country of Japan, such other types of organizations may include ICTRP by WHO, JapicCTI managed by JAPIC, which is a “General Incorporated Foundation” based on the agreement of the Japan Pharmaceutical Manufacturers Association, UMIN-CTR (UMIN Clinical Trials Registry) managed by UMIN (University Hospital Medical Information Network) in Japan, and JMACCT Clinical Trial Registry managed by JMACCT, which is an organization of the Japan Medical Association. Examples of clinical trial data stored in external data source 112 that are not already available to search module 108 include, for each clinical trial, disease specific endpoints, endpoint measurements, and other outcome-related results. Thus, in various embodiments, search module 108 retrieves clinical trial outcome data from external data source 112.

In various embodiments, analysis module 110 performs data processing on the retrieved clinical trial data from external data source 112 in order to provide aggregated results. In some embodiments, analysis module 110 is configured to use a language model based on machine learning techniques to interpret the retrieved clinical trial data and import the interpreted data as structured data. In some embodiments, the machine learning model is used to recognize and extract specific data components (e.g., patient, indication, outcome, phase of trial, compound, cohort, study design, etc.) from the retrieved clinical trial data. Machine learning techniques for data extraction are described in further detail herein. Specifically, in various embodiments, machine learning techniques are utilized to extract outcomes of clinical trials. The extracted outcomes are not ensured to be in a standardized form. Thus, further processing may be required in order to standardize outcomes for comparison purposes. In various embodiments, analysis module 110 is configured to, based on a similarity analysis, match clinical trial endpoints identified in the retrieved clinical trial data to corresponding normalized endpoint options. An example of a user interface element showing a list of normalized endpoint options that a user can select is illustrated in FIG. 6G. Normalized endpoints list 662 provides a list of standardized endpoints for pulmonary arterial hypertension that can be compared to machine learning extracted endpoints from clinical trial data (e.g., obtained from external data source 112). In some embodiments, extracted endpoints are compared to standardized endpoints using a string comparison technique (e.g., word mover distance). Stated alternatively, for each extracted endpoint, a closest standardized endpoint (e.g., one from list 662) can be determined. String comparison techniques to normalize extracted endpoints are described in further detail herein. In various embodiments, the user is able to manually confirm the normalized endpoints that are generated (e.g., by a machine learning model). FIG. 6H illustrates an example of a user interface element in which endpoints are selected and confirmed for clinical trials. In the example illustrated, confirmations 672 and 674 are executed, via confirmation selection tools, to add their corresponding clinical trials to a collection of clinical trials that will ultimately be presented to the user. Accordingly, such an interface may generate one or more confirmation selection tools, wherein actuation of a confirmation selection tool confirms intent to include the corresponding clinical trial. In an embodiment, the confirmation selection tools, as shown in FIG. 6H, may include a drop down menu or other means of manually selecting a desired endpoint.

In various embodiments, analysis module 110 is configured to use the clinical trials that have been selected and confirmed (having extracted endpoints that are matched to normalized endpoint options) to generate an aggregated collection of clinical trial results. In various embodiments, these aggregated results are provided to a user (e.g., by transmitting the results to client device 102 via network 104). An example of aggregated results in tabular form is shown in FIG. 6I. In aggregated results table 682, selected clinical trials (e.g., selected from the interface of FIG. 6H) are represented by the columns and clinical trial properties are represented by the rows. Referring to FIG. 6H, the second column may be the normalized entity column, wherein entries in such a column include the name of the normalized entity corresponding to said clinical trial. Further, the third column may be the similarity score column, wherein entries in such a column include the similarity/match score calculated during the similarity analysis for each of the corresponding entities.

Portions of the communication path between the components are shown. Other communication paths may exist, and the example of FIG. 1 has been simplified to illustrate the example clearly. Although single instances of components have been shown to simplify the diagram, additional instances of any of the components shown in FIG. 1 may exist. For example, additional clients may exist. The number of components and the connections shown in FIG. 1 are merely illustrative. Components not shown in FIG. 1 may also exist.

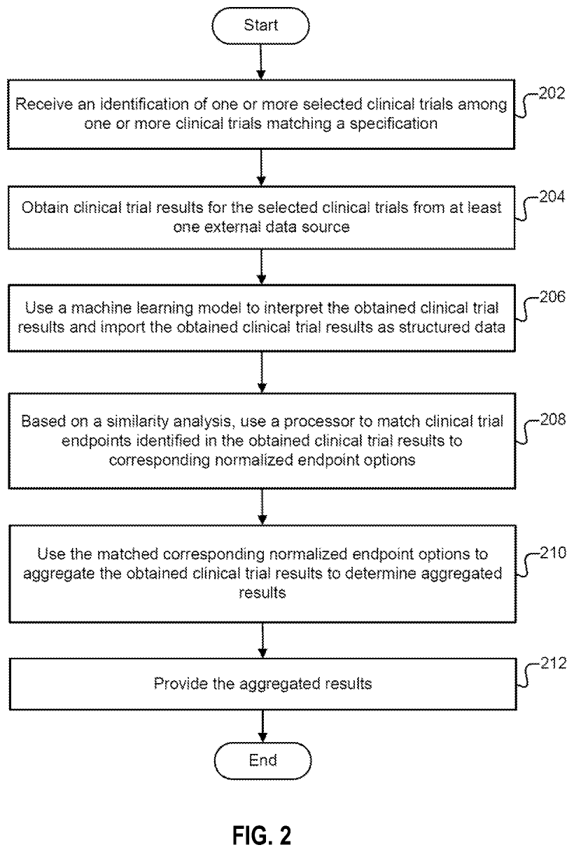

FIG. 2 is a flow diagram illustrating an embodiment of a process for obtaining and aggregating clinical trial results. In some embodiments, the process of FIG. 2 is performed by server 106 of FIG. 1.

At 202, an identification of one or more selected clinical trials among one or more clinical trials matching a specification may be received. In some embodiments, the identification is performed by search module 108. In some embodiments, search module 108 utilizes a separate service (e.g., an online search service with permissions or access to clinical trial summary data) to perform the identification. It is also possible for search module 108 to perform the identification based on clinical trial summary data that is possessed internally. The specification may be comprised of various clinical trial properties, such as a clinical trial identifier, status, phase, title, sponsor, number of sites, enrollment count, intervention, MoA, actions, start date, end date, endpoints, drug category, and so forth. Accordingly, the specification may be a list, string, filter representation, or other structure comprising properties, wherein the specification (and its contained properties) may be customized by user's selections, and wherein the specification may be utilized to identify selected clinical trials among one or more clinical trials. In some embodiments, an initial group of clinical trials is determined based on a first set of properties and the initial group is narrowed through filtering using a second set of properties. The specification may be received from a user via a graphical user interface such as that of FIG. 6D. However, in alternate embodiments, the specification may be directly uploaded to the system, circumnavigating the graphical user interface.

At 204, clinical trial results for the selected clinical trials may be obtained from at least one external data source. In some embodiments, the external data source comprises an online database of clinical trial data maintained by one or more government entities responsible for regulating clinical trials. It is also possible for the online database of clinical trial data to be maintained by other types of organizations, such as one or more international agencies, public agencies such as university network organizations, organizations of medical associations, foundations based on an association of pharmaceutical manufacturers, etc. Specific examples of such other types of organizations are described above. In various embodiments, the clinical trial results are stored in a format (e.g., tabular and/or text-based) that requires additional data extraction and/or processing. It is possible for the clinical trial results to appear in various formats. Example formats include press releases, scientific articles, Food and Drug Administration (FDA) labels, data tables, or other formats. In some embodiments, at 202, clinical trial names corresponding to a disease indication are identified and then clinical trial data corresponding to the clinical trial names are obtained from the external data source. In an embodiment, the method described herein may operate based on obtaining clinical trial results for one selected clinical trial. However, in various embodiments, the method described herein may operate based on obtaining clinical trial results for two or more clinical trials. As a non-limiting example, a user may opt to view a side-by-side comparison of two selected clinical trials, wherein the method may include obtaining clinical trial results for the two selected clinical trials from at least one external data source. In another non-limiting example, a user may opt to view a landscape comparison of trials, wherein the user may select between five and ten clinical trials. In an embodiment, there may exist a hierarchy of external data source sources. In such an embodiment, external data sources comprising robust, structured, or nearly-structured data may be ranked higher in the data source hierarchy. For example, data sources corresponding to government-necessitated clinical trial databases having relatively clean and standardized scientific language may be ranked relatively high on the data source hierarchy, while data sources corresponding to press releases or news articles having rather colloquial or unstructured information may be ranked relatively low on the data source hierarchy. Accordingly, in obtaining clinical trial results from the one or more external data sources, the system may be configured to opt for data sources ranked higher on the data sources hierarchy. In such an embodiment, the system may select data from higher ranking sources, wherein the system may opt for lower ranking sources when said higher ranking sources fail to return the desired clinical trial results. In doing so, the system may optimize performance by streamlining clinical trial results retrieval to those sources most likely to include robust data with a preferred data structure. Thus, the computational burden may be reduced for later steps of normalization and/or entity extraction.

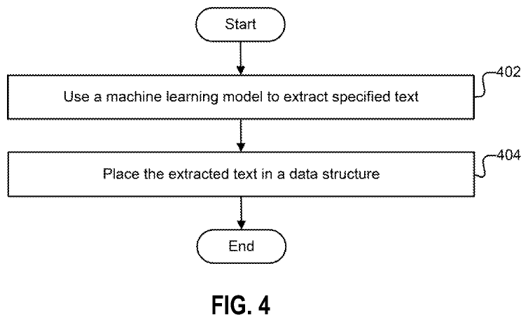

At 206, a machine learning model may be used to interpret the obtained clinical trial results and import the obtained clinical trial results as structured data. For the purposes of this disclosure, “structured data” may refer to data and/or data formats that are constructed from “unstructured data.” Accordingly, structured data may refer to data that has been processed, categorized, or otherwise arranged, for example, in a format conducive for later data aggregation or visualization. Thus, unstructured data may refer to narrative form text. In some embodiments, the machine learning model is a predictive model that has been trained to locate and extract specified types of words in unstructured textual data and classify them into pre-defined categories. In some embodiments, the pre-defined categories are related to disease specific endpoints, endpoint measurements, and other outcome-related data. In various embodiments, the extracted words are placed in a data structure with a specified format. Machine learning-based information extraction and importation of clinical trial results is described in further detail below (e.g., see FIG. 4). In one embodiment, the system may include a plurality of machine learning models or tailored instances of a single machine learning model, wherein each machine learning model or tailored instance may be configured to interpret data from a particular source. In such an embodiment, the utilization of a particular machine learning model or tailored instance may correspond to the data source hierarchy ranking of the corresponding obtained clinical trial results. For example, a first machine learning model may be equipped for entity extraction of low-ranking data sources, wherein a robust entity extraction model is preferred to interpret texts written with a high degree of freedom (e.g., less standardized technical language). Similarly, for example, a second machine learning model, having a nimble or slim framework, may be equipped to extract entities from highly technical and structured texts (e.g., government-necessitated clinical trial reporting databases).

At 208, based on a similarity analysis, a processor may be used to match clinical trial endpoints identified in the obtained clinical trial results to corresponding normalized endpoint options. In some embodiments, the similarity analysis includes comparing the clinical trial endpoints identified in the obtained clinical trial results with standardized endpoints from a list of endpoint options based on a word distance metric. Various types of word distance metrics may be utilized when comparing vectorized representations, including Euclidean distance, Hamming distance, Levenshtein distance, and cosine similarity. In an embodiment, a word distance metric tool may be utilized, wherein distance between two texts, syntactic units, or documents is evaluated, even in scenarios where no common keywords exist. In such an embodiment, a word distance metric tool may utilize vector embeddings of words. As a non-limiting example, the word distance metric tool may determine the distance between various texts by evaluating the cumulative distance to move all words in a first text to match a second text. Accordingly, the word distance metric tool may be configured to utilize an optimal transport formulation in view of the underlying geometry of the word space of one or more clinical trial results. In various embodiments, each identified clinical trial endpoint is normalized to a standardized endpoint option by mapping the identified clinical trial endpoint to a closest standardized endpoint option in terms of a specified word distance metric. Examples of standardized endpoint options for a specific disease indication are shown in FIG. 6G.

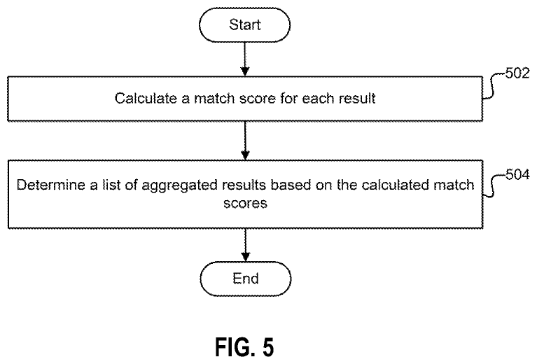

At 210, the matched corresponding normalized endpoint options may be used to aggregate the obtained clinical trial results to determine aggregated results. In various embodiments, clinical trials in which clinical trial endpoints have been mapped to one or more specific standardized endpoint options are grouped together. For example, with respect to the example of pulmonary arterial hypertension, clinical trials that have a machine learning determined endpoint that is mapped to the standardized endpoint “Assessment of Six Minute Walk Test” may be grouped together. Within such a group, an ordered list of clinical trials may be generated based on match scores of the clinical trials (e.g., see FIG. 5). In various embodiments, data corresponding to the group of clinical trials is aggregated and placed in a tabular format (e.g., an Excel tabular format). In the tabular format, columns of a table may correspond to different clinical trials and rows may correspond to various properties (e.g., endpoints and outcomes) of the different clinical trials. An example tabular format with aggregated clinical trial results is illustrated in FIG. 6I. In an embodiment, the aggregation of the obtained clinical trial results to determine aggregated results may be based on the matched corresponding normalized endpoints and/or confirmation of said normalized endpoints. For example, the aggregation of determined aggregated results may take into account a user's input (i.e., confirmation of desired normalized endpoints), in addition to the matched corresponding endpoint options. In such a non-limiting example, a secondary basis (e.g., the user's confirmation input) may minimize the likelihood that an erroneous matched endpoint advances to the aggregated results.

At 212, the aggregated results may be provided. In some embodiments, the aggregated results are reported to a user that requested the aggregated results. For example, the user may be a person utilizing client device 102 to request aggregated clinical trial results from server 106.

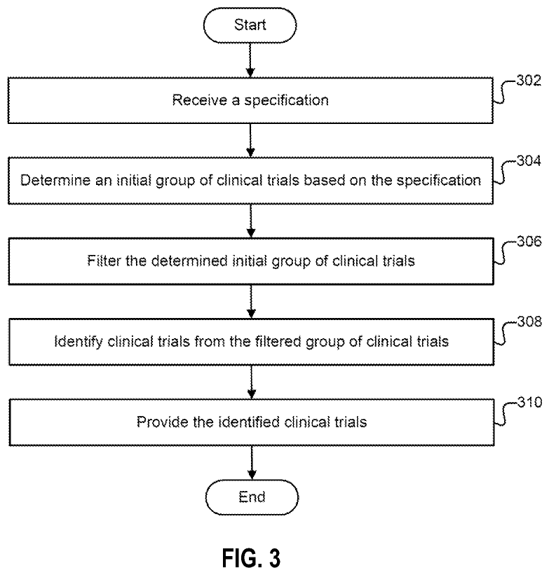

FIG. 3 is a flow diagram illustrating an embodiment of a process for identifying clinical trials matching a specification. In some embodiments, the process of FIG. 3 provides an output received at 202 of FIG. 2. In some embodiments, the process of FIG. 3 is performed by search module 108.

At 302, a specification may be received. In some embodiments, the specification includes a therapeutic area (e.g., cardiovascular disease as shown in FIG. 6A) and a disease indication within the therapeutic area (e.g., pulmonary arterial hypertension within cardiovascular disease as shown in FIG. 6B). The specification may also include various clinical trial properties, such as a clinical trial identifier, status, phase, title, sponsor, number of sites, enrollment count, intervention, MoA, actions, start date, end date, endpoints, drug category, and so forth. In various embodiments, the specification is received from a user (e.g., via client device 102).

At 304, an initial group of clinical trials may be determined based on the specification. For example, if the specification includes cardiovascular disease as a therapeutic area, pulmonary arterial hypertension as a specific disease indication within the therapeutic area, and completed phase 3 as criteria, then the initial group of clinical trials would be completed phase 3 clinical trials that target the cardiovascular disease pulmonary arterial hypertension.

At 306, the determined initial group of clinical trials may be filtered. In some embodiments, the initial group of clinical trials is narrowed based on other properties. It is also possible for these other properties to have already been applied to arrive at the initial group of clinical trials, in which case the initial group of clinical trials may already be sufficiently narrow in scope. In some embodiments, a user filters the initial group of clinical trials via a user interface (e.g., see FIG. 6D).

At 308, clinical trials may be identified from the filtered group of clinical trials. In some embodiments, identifying the clinical trials includes presenting the filtered group of clinical trials to a user via a user interface to confirm the presented filtered group of clinical trials.

At 310, the identified clinical trials may be provided. In some embodiments, the identified clinical trials are provided as a list of clinical trials for further processing. In a further embodiment, the list identifies which of the one or more clinical trials matching the specification include clinical result data obtainable from the at least one external data source. For example, the list may include clinical trials where clinical trial results may be obtained but have not yet been obtained. Thus, the list may decrease required bandwidth by not populating the clinical results initially, but instead allowing a user or the system to select which clinical trial results should be called upon, generated, and/or displayed.

FIG. 4 is a flow diagram illustrating an embodiment of a process for importing clinical trial results as structured data. In some embodiments, the process of FIG. 4 is performed by analysis module 110. In some embodiments, at least a portion of the process of FIG. 4 is performed in 206.

At 402, a machine learning model may be used to extract specified text. In various embodiments, the specified text is extracted from a larger collection of text in which the specified text does not appear in a standardized location, layout, and/or pattern. The larger collection of text may be comprised of a block of text with tables or other structures. In various embodiments, the specified text includes words that belong to pre-defined categories. An example of a pre-defined category is clinical endpoints. In various embodiments, the machine learning model has been trained on training samples. For example, when the machine learning model is configured to extract clinical endpoints, it would have been trained on text in which various types of clinical endpoints appear in training text. Stated alternatively, in various embodiments, the machine learning model has been trained on datasets comprising a constrained set of collections of text with prescribed clinical endpoint categories to which extracted clinical endpoints identified from obtained clinical trial results belong. It is also possible to train the machine learning model to extract other types of words. For example, with respect to clinical trials, the machine learning model can also be trained to recognize patient characteristics, indications, outcomes, phases, drug names, compounds, cohorts, study design characteristics, and so forth. In some embodiments, the machine learning model is a named entity recognition (NER) model. In some embodiments, the NER model utilizes a recurrent neural network (RNN) architecture, such as Long Short-Term Memory (LSTM), bidirectional LSTM, or gated recurrent unit (GRU) structures. Other machine learning approaches are also possible, e.g., using convolutional neural networks (CNNs) or conditional random fields (CRFs). In addition to PICO entity types (e.g., outcome, intervention, and outcome group), the NER model may be configured to extract entities based on any of the follow non-limiting entity categories: drug use, dosage, frequency, duration, indication, time frame, measurement, and modifier. Accordingly, by configuring the NER model with the aforementioned entity types, the machine learning model may have a broader understanding of entity categories (e.g., those related to clinical trial texts) and may improve the subsequent clinical trial data analysis. Thus, by training the machine learning model on a constrained set of collection of texts with prescribed endpoint categories, the machine learning model may exhibit increased efficacy when interpreting clinical trial results. However, as contemplated by a person of ordinary skill in the art, the NER model may be adapted to extract entities based on any suitable entity categories.

At 404, the extracted text may be placed in a data structure. In some embodiments, the data structure includes various fields that correspond to various types of extracted text. For example, one or more endpoint fields may be used to store one or more corresponding endpoints that are extracted. In various embodiments, each field has a title associated with that field, e.g., “Endpoint” or “Outcome” for a field that stores an extracted clinical endpoint.

FIG. 5 is a flow diagram illustrating an embodiment of a process for determining aggregated results. In some embodiments, the process of FIG. 5 is performed by analysis module 110. In some embodiments, at least a portion of the process of FIG. 5 is performed in 210.

At 502, a match score may be calculated for each result. In various embodiments, each result is a word or phrase extracted by a trained machine learning model that has been converted to a standardized form. For the purposes of this disclosure, words, phrases, sentences, or other segments of text may be referred to herein as syntactic units. For example, the word or phrase may be what the machine learning model recognizes as an endpoint. For example, the extracted phrase may be “Six Minute Test” and the standardized form may be “Assessment of Six Minute Walk Test”. The match score compares the quality of the match, e.g., between “Six Minute Test” and “Assessment of Six Minute Walk Test”. In some embodiments, the match score comprises a word distance metric, e.g., Euclidean distance, Hamming distance, Levenshtein distance, cosine distance, or another metric. In various embodiments, a higher match score indicates closer distance (higher similarity) between the extracted phrase and the standardized form to which it is matched.

At 504, a list of aggregated results is determined based on the calculated match scores. For example, the list may be in descending order from highest calculated match score to lowest calculated match score.

FIGS. 6A-6I illustrate various user interface elements of a system for obtaining and aggregating clinical trial results. FIGS. 6A-6I are described above with respect to abovementioned FIGS. 2-5.

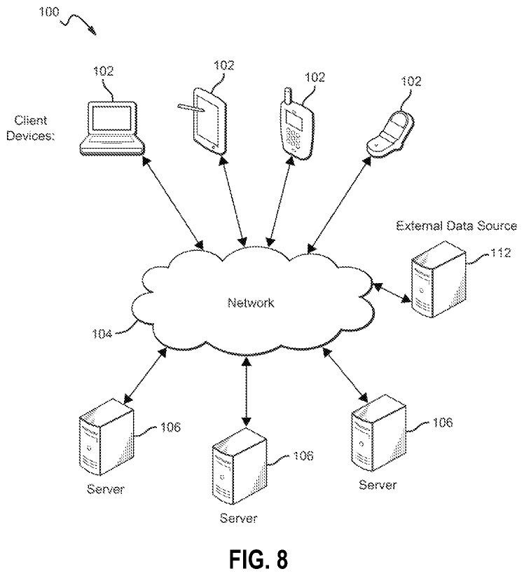

FIG. 8 is a functional diagram illustrating a programmed computer system. In some embodiments, the processes of FIGS. 2-5 are executed by computer system 700. In some embodiments, search module 108 and/or analysis module 110 of FIG. 1 are embodied in computer program instructions that are executed by computer system 700.

In the example shown, computer system 700 includes various subsystems as described below. Computer system 700 includes at least one microprocessor subsystem (also referred to as a processor or a central processing unit (CPU)) 702. Computer system 700 can be physical or virtual (e.g., a virtual machine). For example, processor 702 can be implemented by a single-chip processor or by multiple processors. In some embodiments, processor 702 is a general-purpose digital processor that controls the operation of computer system 700. Using instructions retrieved from memory 730, processor 702 controls the reception and manipulation of input data, and the output and display of data on output devices.

Processor 702 is coupled bi-directionally with memory 730, which can include a first primary storage, typically a random-access memory (RAM), and a second primary storage area, typically a read-only memory (ROM). As is well known in the art, primary storage can be used as a general storage area and as scratch-pad memory, and can also be used to store input data and processed data. Primary storage can also store programming instructions and data, in the form of data objects and text objects, in addition to other data and instructions for processes operating on processor 702. Also, as is well known in the art, primary storage typically includes basic operating instructions, program code, data, and objects used by processor 702 to perform its functions (e.g., programmed instructions). For example, memory 730 can include any suitable computer-readable storage media, described below, depending on whether, for example, data access needs to be bidirectional or uni-directional. For example, processor 702 can also directly and very rapidly retrieve and store frequently needed data in a cache memory (not shown).

Network interface 714 allows processor 702 to be coupled to another computer, computer network, or telecommunications network using a network connection as shown. For example, through network interface 714, processor 702 can receive information (e.g., data objects or program instructions) from another network or output information to another network in the course of performing method/process steps. Information, often represented as a sequence of instructions to be executed on a processor, can be received from and outputted to another network. An interface card or similar device and appropriate software implemented by (e.g., executed/performed on) processor 702 can be used to connect computer system 700 to an external network and transfer data according to standard protocols. Processes can be executed on processor 702, or can be performed across a network such as the Internet, intranet networks, or local area networks, in conjunction with a remote processor that shares a portion of the processing. Additional mass storage devices (not shown) can also be connected to processor 702 through network interface 714.

An auxiliary I/O device interface (not shown) can be used in conjunction with computer system 700. The auxiliary I/O device interface can include general and customized interfaces that allow processor 702 to send and, more typically, receive data from other devices such as microphones, touch-sensitive displays, transducer card readers, tape readers, voice or handwriting recognizers, biometrics readers, cameras, portable mass storage devices, and other computers.

In addition, various embodiments disclosed herein further relate to computer storage products with a computer readable medium that includes program code for performing various computer-implemented operations. The computer-readable medium is any data storage device that can store data which can thereafter be read by a computer system. Examples of computer-readable media include, but are not limited to, all the media mentioned above: magnetic media such as hard disks, floppy disks, and magnetic tape; optical media such as CD-ROM disks; magneto-optical media such as optical disks; and specially configured hardware devices such as application-specific integrated circuits (ASICs), programmable logic devices (PLDs), and ROM and RAM devices. Examples of program code include both machine code, as produced, for example, by a compiler, or files containing higher level code (e.g., script) that can be executed using an interpreter.

The computer system shown in FIG. 8 is but an example of a computer system suitable for use with the various embodiments disclosed herein. Other computer systems suitable for such use can include additional or fewer subsystems. In addition, bus is illustrative of any interconnection scheme serving to link the subsystems. Other computer architectures having different configurations of subsystems can also be utilized.

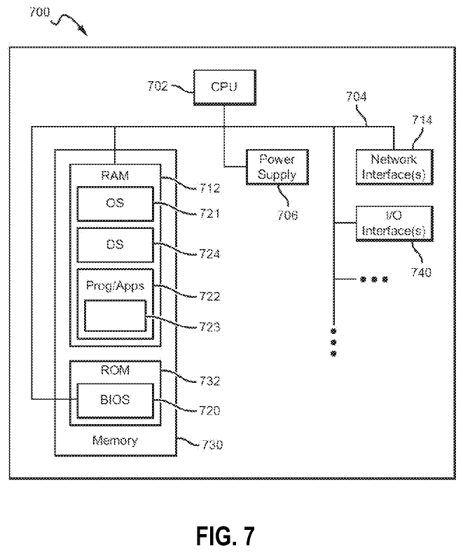

FIG. 7 illustrates a block diagram of an computer system 700 that can implement one or more aspects of an apparatus, system and method for validating and correcting user information (the “Engine”) according to one embodiment of the invention. Instances of the electronic device 200 may include servers, e.g., servers 106, and client devices, e.g., client devices 102. In general, the computer system 700 can include a processor/CPU 702, memory 730, a power supply 706, and input/output (I/O) components/devices 740, e.g., microphones, speakers, displays, touchscreens, keyboards, mice, keypads, microscopes, GPS components, cameras, heart rate sensors, light sensors, accelerometers, targeted biometric sensors, etc., which may be operable, for example, to provide graphical user interfaces or text user interfaces.

A user may provide input via a touchscreen of an computer system 700. A touchscreen may determine whether a user is providing input by, for example, determining whether the user is touching the touchscreen with a part of the user's body such as his or her fingers. The computer system 700 can also include a communications bus 704 that connects the aforementioned elements of the computer system 700. Network interfaces 714 can include a receiver and a transmitter (or transceiver), and one or more antennas for wireless communications.

The processor 702 can include one or more of any type of processing device, e.g., a Central Processing Unit (CPU), and a Graphics Processing Unit (GPU). Also, for example, the processor can be central processing logic, or other logic, may include hardware, firmware, software, or combinations thereof, to perform one or more functions or actions, or to cause one or more functions or actions from one or more other components. Also, based on a desired application or need, central processing logic, or other logic, may include, for example, a software-controlled microprocessor, discrete logic, e.g., an Application Specific Integrated Circuit (ASIC), a programmable/programmed logic device, memory device containing instructions, etc., or combinatorial logic embodied in hardware. Furthermore, logic may also be fully embodied as software.

The memory 730, which can include Random Access Memory (RAM) 712 and Read Only Memory (ROM) 732, can be enabled by one or more of any type of memory device, e.g., a primary (directly accessible by the CPU) or secondary (indirectly accessible by the CPU) storage device (e.g., flash memory, magnetic disk, optical disk, and the like). The RAM can include an operating system 721, data storage 724, which may include one or more databases, and programs and/or applications 722, which can include, for example, software aspects of the program 723. The ROM 732 can also include Basic Input/Output System (BIOS) 720 of the electronic device.

Persistent memory (e.g., a removable mass storage device) provides additional data storage capacity for computer system 700, and is coupled either bi-directionally (read/write) or uni-directionally (read only) to processor 702. For example, persistent memory can also include computer-readable media such as magnetic tape, flash memory, PC-CARDS, portable mass storage devices, holographic storage devices, and other storage devices. A fixed mass storage can also, for example, provide additional data storage capacity. The most common example of fixed mass storage 720 is a hard disk drive. Persistent memory and fixed mass storage generally store additional programming instructions, data, and the like that typically are not in active use by the processor 702. It will be appreciated that the information retained within persistent memory and fixed mass storage can be incorporated, if needed, in standard fashion as part of memory (e.g., RAM) as virtual memory.

In addition to providing processor 702 access to storage subsystems, bus can also be used to provide access to other subsystems and devices. As shown, these can include a display monitor, a network interface 714, a keyboard, and a pointing device, as well as an auxiliary input/output device interface, a sound card, speakers, and other subsystems as needed. For example, pointing device can be a mouse, stylus, track ball, or tablet, and is useful for interacting with a graphical user interface.

Software aspects of the program 723 are intended to broadly include or represent all programming, applications, algorithms, models, software and other tools necessary to implement or facilitate methods and systems according to embodiments of the invention. The elements may exist on a single computer or be distributed among multiple computers, servers, devices or entities.

The power supply 706 contains one or more power components, and facilitates supply and management of power to the computer system 700.

The input/output components, including Input/Output (I/O) components/devices 740 interfaces, can include, for example, any interfaces for facilitating communication between any components of the computer system 700, components of external devices (e.g., components of other devices of the network or system 100), and end users. For example, such components can include a network card that may be an integration of a receiver, a transmitter, a transceiver, and one or more input/output interfaces. A network card, for example, can facilitate wired or wireless communication with other devices of a network. In cases of wireless communication, an antenna can facilitate such communication. Also, some of the input/output components/devices 740 interfaces and the bus 704 can facilitate communication between components of the computer system 700, and in an example can ease processing performed by the processor 702.

Where the computer system 700 is a server, it can include a computing device that can be capable of sending or receiving signals, e.g., via a wired or wireless network, or may be capable of processing or storing signals, e.g., in memory as physical memory states. The server may be an application server that includes a configuration to provide one or more applications, e.g., aspects of the Engine, via a network to another device. Also, an application server may, for example, host a web site that can provide a user interface for administration of example aspects of the Engine.

Any computing device capable of sending, receiving, and processing data over a wired and/or a wireless network may act as a server, such as in facilitating aspects of implementations of the Engine. Thus, devices acting as a server may include devices such as dedicated rack-mounted servers, desktop computers, laptop computers, set top boxes, integrated devices combining one or more of the preceding devices, and the like.

Servers may vary widely in configuration and capabilities, but they generally include one or more central processing units, memory, mass data storage, a power supply, wired or wireless network interfaces, input/output interfaces, and an operating system such as Windows Server, Mac OS X, Unix, Linux, FreeBSD, and the like.

A server may include, for example, a device that is configured, or includes a configuration, to provide data or content via one or more networks to another device, such as in facilitating aspects of an example apparatus, system and method of the Engine. One or more servers may, for example, be used in hosting a Web site, such as the web site www.microsoft.com. One or more servers may host a variety of sites, such as, for example, business sites, informational sites, social networking sites, educational sites, wikis, financial sites, government sites, personal sites, and the like.

Servers may also, for example, provide a variety of services, such as Web services, third-party services, audio services, video services, email services, HTTP or HTTPS services, Instant Messaging (IM) services, Short Message Service (SMS) services, Multimedia Messaging Service (MMS) services, File Transfer Protocol (FTP) services, Voice Over IP (VOIP) services, calendaring services, phone services, and the like, all of which may work in conjunction with example aspects of an example systems and methods for the apparatus, system and method embodying the Engine. Content may include, for example, text, images, audio, video, and the like.

In example aspects of the apparatus, system and method embodying the Engine, client devices may include, for example, any computing device capable of sending and receiving data over a wired and/or a wireless network. Such client devices may include desktop computers as well as portable devices such as cellular telephones, smart phones, display pagers, Radio Frequency (RF) devices, Infrared (IR) devices, Personal Digital Assistants (PDAs), handheld computers, GPS-enabled devices tablet computers, sensor-equipped devices, laptop computers, set top boxes, wearable computers such as the Apple Watch and Fitbit, integrated devices combining one or more of the preceding devices, and the like.

Client devices such as client devices 102, as may be used in an example apparatus, system and method embodying the Engine, may range widely in terms of capabilities and features. For example, a cell phone, smart phone or tablet may have a numeric keypad and a few lines of monochrome Liquid-Crystal Display (LCD) display on which only text may be displayed. In another example, a Web-enabled client device may have a physical or virtual keyboard, data storage (such as flash memory or SD cards), accelerometers, gyroscopes, respiration sensors, body movement sensors, proximity sensors, motion sensors, ambient light sensors, moisture sensors, temperature sensors, compass, barometer, fingerprint sensor, face identification sensor using the camera, pulse sensors, heart rate variability (HRV) sensors, beats per minute (BPM) heart rate sensors, microphones (sound sensors), speakers, GPS or other location-aware capability, and a 2D or 3D touch-sensitive color screen on which both text and graphics may be displayed. In some embodiments multiple client devices may be used to collect a combination of data. For example, a smart phone may be used to collect movement data via an accelerometer and/or gyroscope and a smart watch (such as the Apple Watch) may be used to collect heart rate data. The multiple client devices (such as a smart phone and a smart watch) may be communicatively coupled.

Client devices, such as client devices 102, for example, as may be used in an example apparatus, system and method implementing the Engine, may run a variety of operating systems, including personal computer operating systems such as Windows, iOS or Linux, and mobile operating systems such as iOS, Android, Windows Mobile, and the like. Client devices may be used to run one or more applications that are configured to send or receive data from another computing device. Client applications may provide and receive textual content, multimedia information, and the like. Client applications may perform actions such as browsing webpages, using a web search engine, interacting with various apps stored on a smart phone, sending and receiving messages via email, SMS, or MIMS, playing games (such as fantasy sports leagues), receiving advertising, watching locally stored or streamed video, or participating in social networks.

In example aspects of the apparatus, system and method implementing the Engine, one or more networks, such as network 104, for example, may couple servers and client devices with other computing devices, including through wireless network to client devices. A network may be enabled to employ any form of computer readable media for communicating information from one electronic device to another. The computer readable media may be non-transitory. Thus, in various embodiments, a non-transitory computer readable medium may comprise instructions stored thereon that, when executed by a processing device, cause the processing device to carry out an operation (e.g., entity extraction and clinical result aggregation). In such an embodiment, the operation may be carried out on a singular device or between multiple devices (e.g., a server and a client device). A network may include the Internet in addition to Local Area Networks (LANs), Wide Area Networks (WANs), direct connections, such as through a Universal Serial Bus (USB) port, other forms of computer-readable media (computer-readable memories), or any combination thereof. On an interconnected set of LANs, including those based on differing architectures and protocols, a router acts as a link between LANs, enabling data to be sent from one to another.

Communication links within LANs may include twisted wire pair or coaxial cable, while communication links between networks may utilize analog telephone lines, cable lines, optical lines, full or fractional dedicated digital lines including T1, T2, T3, and T4, Integrated Services Digital Networks (ISDNs), Digital Subscriber Lines (DSLs), wireless links including satellite links, optic fiber links, or other communications links known to those skilled in the art. Furthermore, remote computers and other related electronic devices could be remotely connected to either LANs or WANs via a modem and a telephone link.

A wireless network, such as wireless network 104, as in an example apparatus, system and method implementing the Engine, may couple devices with a network. A wireless network may employ stand-alone ad-hoc networks, mesh networks, Wireless LAN (WLAN) networks, cellular networks, and the like.

A wireless network may further include an autonomous system of terminals, gateways, routers, or the like connected by wireless radio links, or the like. These connectors may be configured to move freely and randomly and organize themselves arbitrarily, such that the topology of wireless network may change rapidly. A wireless network may further employ a plurality of access technologies including 2nd (2G), 3rd (3G), 4th (4G), 5th (5G) generation, Long Term Evolution (LTE) radio access for cellular systems, WLAN, Wireless Router (WR) mesh, and the like. Access technologies such as 2G, 2.5G, 3G, 4G, 5G, and future access networks may enable wide area coverage for client devices, such as client devices with various degrees of mobility. For example, a wireless network may enable a radio connection through a radio network access technology such as Global System for Mobile communication (GSM), Universal Mobile Telecommunications System (UMTS), General Packet Radio Services (GPRS), Enhanced Data GSM Environment (EDGE), 3GPP Long Term Evolution (LTE), LTE Advanced, Wideband Code Division Multiple Access (WCDMA), Bluetooth, 702.11b/g/n, and the like. A wireless network may include virtually any wireless communication mechanism by which information may travel between client devices and another computing device, network, and the like.

Internet Protocol (IP) may be used for transmitting data communication packets over a network of participating digital communication networks, and may include protocols such as TCP/IP, UDP, DECnet, NetBEUI, IPX, Appletalk, and the like. Versions of the Internet Protocol include IPv4 and IPv6. The Internet includes local area networks (LANs), Wide Area Networks (WANs), wireless networks, and long-haul public networks that may allow packets to be communicated between the local area networks. The packets may be transmitted between nodes in the network to sites each of which has a unique local network address. A data communication packet may be sent through the Internet from a user site via an access node connected to the Internet. The packet may be forwarded through the network nodes to any target site connected to the network provided that the site address of the target site is included in a header of the packet. Each packet communicated over the Internet may be routed via a path determined by gateways and servers that switch the packet according to the target address and the availability of a network path to connect to the target site.

The header of the packet may include, for example, the source port (16 bits), destination port (16 bits), sequence number (32 bits), acknowledgement number (32 bits), data offset (4 bits), reserved (6 bits), checksum (16 bits), urgent pointer (16 bits), options (variable number of bits in multiple of 8 bits in length), padding (may be composed of all zeros and includes a number of bits such that the header ends on a 32 bit boundary). The number of bits for each of the above may also be higher or lower.

A “content delivery network” or “content distribution network” (CDN), as may be used in an example apparatus, system and method implementing the Engine, generally refers to a distributed computer system that comprises a collection of autonomous computers linked by a network or networks, together with the software, systems, protocols and techniques designed to facilitate various services, such as the storage, caching, or transmission of content, streaming media and applications on behalf of content providers. Such services may make use of ancillary technologies including, but not limited to, “cloud computing,” distributed storage, DNS request handling, provisioning, data monitoring and reporting, content targeting, personalization, and business intelligence. A CDN may also enable an entity to operate and/or manage a third party's web site infrastructure, in whole or in part, on the third party's behalf.

A Peer-to-Peer (or P2P) computer network relies primarily on the computing power and bandwidth of the participants in the network rather than concentrating it in a given set of dedicated servers. P2P networks are typically used for connecting nodes via largely ad hoc connections. A pure peer-to-peer network does not have a notion of clients or servers, but only equal peer nodes that simultaneously function as both “clients” and “servers” to the other nodes on the network.

Embodiments of the present invention include apparatuses, systems, and methods implementing the Engine. Embodiments of the present invention may be implemented on one or more of client devices 102, which are communicatively coupled to servers including servers 106. Moreover, client devices 102 may be communicatively (wirelessly or wired) coupled to one another. In particular, software aspects of the Engine may be implemented in the program 223. The program 723 may be implemented on one or more client devices 102, one or more servers 106 or a combination of one or more client devices 102 and one or more servers 106.

In an embodiment, the system may receive, process, generate and/or store time series data. The system may include an application programming interface (API). The API may include an API subsystem. The API subsystem may allow a data source to access data. The API subsystem may allow a third-party data source to send the data. In one example, the third-party data source may send JavaScript Object Notation (“JSON”)-encoded object data. In an embodiment, the object data may be encoded as XML-encoded object data, query parameter encoded object data, or byte-encoded object data.

FIG. 8 illustrates components of one embodiment of an environment in which the invention may be practiced. Not all of the components may be required to practice the invention, and variations in the arrangement and type of the components may be made without departing from the spirit or scope of the invention. As shown, the system 100 includes one or more Local Area Networks (“LANs”)/Wide Area Networks (“WANs”) 104, one or more wireless networks 104, one or more wired or wireless client devices 102, mobile or other wireless client devices 102, servers 106, and may include or communicate with one or more data stores or databases. Various of the client devices 102 may include, for example, desktop computers, laptop computers, set top boxes, tablets, cell phones, smart phones, smart speakers, wearable devices (such as the Apple Watch) and the like. Servers 106 can include, for example, one or more application servers, content servers, search servers, and the like. FIG. 8 also illustrates external data source 112.

Illustrative examples of the technologies disclosed herein are provided below. An embodiment of the technologies may include any one or more, and any combination of, the examples described below.

Example 1 includes a method, the method comprising the steps of: receiving one or more selected clinical trials, wherein the one or more selected clinical trials match a specification; obtaining clinical trial results for the one or more selected clinical trials from at least one external data source; interpreting, via a machine learning model, the obtained clinical trial results; importing the obtained clinical trial results as structured data; matching, based on a similarity analysis, via a processor, clinical trial endpoints identified in the obtained clinical trial results to corresponding normalized endpoint options; aggregating, based on the matched corresponding normalized endpoint options, the obtained clinical trial results to determine aggregated results; and providing the aggregated results.

Example 2 includes the subject matter of Example 1, and wherein the identification of the one or more selected clinical trials is provided from a list of the one or more clinical trials matching the specification.

Example 3 includes the subject matter of Example 2, and wherein the list of the one or more clinical trials matching the specification identifies which ones of the one or more clinical trials matching the specification have clinical result data obtainable from the at least one external data source.

Example 4 includes the subject matter of any of Examples 1-3, and wherein the specification is a search specification including a disease category.

Example 5 includes the subject matter of Example 4, and wherein the search specification further includes a specific disease of the disease category.

Example 6 includes the subject matter of any of Examples 1-5, and wherein the specification is a search specification including a clinical trial phase category.

Example 7 includes the subject matter of any of Examples 1-6, and further comprising requesting a user to provide the specification using a graphical user interface.

Example 8 includes the subject matter of any of Examples 1-7, and wherein the at least one external data source includes an online database of clinical trial data maintained by one or more government entities responsible for regulating clinical trials, international agencies, university network organizations, organizations of medical associations, or foundations based on an association of pharmaceutical manufacturers.

Example 9 includes the subject matter of any of Examples 1-8, and wherein the machine learning model includes a named entity recognition (NER) model.

Example 10 includes the subject matter of Example 9, and wherein the NER model utilizes a recurrent neural network (RNN) architecture.

Example 11 includes the subject matter of any of Examples 1-10, and wherein using the machine learning model to interpret the obtained clinical trial results includes automatically extracting specified text from unstructured text of the obtained clinical trial results.

Example 12 includes the subject matter of any of Examples 1-11, and wherein the structured data includes a collection of fields corresponding to categories of syntactic units (e.g., words or phrases) extracted from the obtained clinical trial results.

Example 13 includes the subject matter of any of Examples 1-12, and wherein the similarity analysis includes computation of a word distance metric between the clinical trial endpoints identified in the obtained clinical trial results and the corresponding normalized endpoint options.

Example 14 includes the subject matter of any of Examples 1-13, and further comprising requesting a user to confirm the matched corresponding normalized endpoint options.

Example 15 includes the subject matter of any of Examples 1-14, and wherein the aggregated results are ordered based on a match score of each result of the aggregated results.

Example 16 includes the subject matter of any of Examples 1-15, and wherein the provided aggregated results have a tabular structure in which different columns of the tabular structure represent different clinical trials and different rows of the tabular structure represent different clinical trial properties.

Example 17 includes the subject matter of any of Examples 1-16, and further comprising one or more selected from the group comprised of filtering, machine translating, and standardizing terminology of the selected clinical trials before obtaining the clinical trial results from the at least one external data source.

Example 18 includes the subject matter of any of Examples 1-17, and wherein the machine learning model has been trained on datasets comprising a constrained set of collections of text with prescribed clinical endpoint categories to which the clinical trial endpoints identified in the obtained clinical trial results belong.

Example 19 includes a system comprising: one or more processors and a memory coupled to at least one of the one or more processors and configured to provide at least one of the one or more processors with instructions for performing the method of any of Examples 1-18.

Example 20 includes a computer program product embodied in a non-transitory computer readable medium and comprising computer instructions for performing the method of any of Examples 1-18.

In an aspect of this disclosure, a computer-implemented method, comprising the steps of receiving one or more selected clinical trials, wherein the one or more selected clinical trials match a specification; and obtaining clinical trial results for the one or more selected clinical trials from at least one external data source. In an embodiment, the method further comprises the steps of interpreting, via a machine learning model, the obtained clinical trial results; importing the obtained clinical trial results as structured data; and matching, based on a similarity analysis, via a processor, clinical trial endpoints identified in the obtained clinical trial results to corresponding normalized endpoint options. In yet a further embodiment, the method may comprise the steps of aggregating, based on the matched corresponding normalized endpoint options, the obtained clinical trial results to determine aggregated results; and providing the aggregated results.

In an embodiment, the one or more selected clinical trials are provided from a list comprising one or more clinical trials matching the specification. In a further embodiment, the list identifies which of the one or more clinical trials matching the specification include clinical result data obtainable from the at least one external data source. The specification may include a disease category. Moreover, the specification may include a specific disease of the disease category. Yet further, the specification may include a clinical trial phase category.

The method may further comprise the step of receiving, from a user, the specification via a graphical user interface. In an embodiment, the at least one external data source comprises an online database of clinical trial data maintained by one or more government entities responsible for regulating clinical trials, international agencies, university network organizations, organizations of medical associations, or foundations based on an association of pharmaceutical manufacturers. The machine learning model may include a named entity recognition (NER) model. Further, the NER model may utilize a recurrent neural network (RNN) architecture.

In an embodiment, interpreting, via the machine learning model, the obtained clinical trial results further comprises automatically extracting specified text from unstructured text of the obtained clinical trial results. The structured data may comprise a collection of fields corresponding to categories of syntactic units extracted from the obtained clinical trial results.

In another embodiment, the similarity analysis comprises computation of a word distance metric between the clinical trial endpoints identified in the obtained clinical trial results and the corresponding normalized endpoint options. The method may further comprise the steps of generating one or more confirmation selection tools, the one or more confirmation selection tools corresponding to the matched corresponding normalized endpoint options; and receiving, from a user, actuation of one or more of the one or more confirmation selection tools.

In an embodiment, the aggregated results are ordered or ranked based on a match score of each result of the aggregated results. The provided aggregated results may comprise a tabular structure comprising one or more columns and one or more rows, and wherein the one or more columns may represent different clinical trials and the one or more rows may represent different clinical trial properties. The method may further comprise one or more selected from the group comprised of: filtering, machine translating, and standardizing terminology of the selected clinical trials before obtaining the clinical trial results from the at least one external data source.

In yet a further embodiment, the machine learning model has been trained on training datasets comprising a constrained set of collections of text with prescribed clinical endpoint categories to which the clinical trial endpoints identified in the obtained clinical trial results belong.

The invention of the present disclosure may be a system, comprising a server comprising at least one server processor, at least one server database, at least one server memory comprising computer-executable server instructions which, when executed by the at least one server processor, cause the server to receive one or more selected clinical trials, wherein the one or more selected clinical trials match a specification; and obtain clinical trial results for the one or more selected clinical trials from at least one external data source. The computer-executable server instructions which, when executed by the at least one server processor, may cause the server to interpret, via a machine learning model, the obtained clinical trial results; import the obtained clinical trial results as structured data; and match, based on a similarity analysis, via a processor, clinical trial endpoints identified in the obtained clinical trial results to corresponding normalized endpoint options. In a further embodiment, the computer-executable server instructions which, when executed by the at least one server processor, cause the server to aggregate, based on the matched corresponding normalized endpoint options, the obtained clinical trial results to determine aggregated results; and provide the aggregated results. In a further embodiment, the system comprises a client device comprising at least one device processor, at least one display, at least one device memory comprising computer-executable device instructions which, when executed by the at least one device processor, cause the client device to receive, from the client device, the specification via a graphical user interface.

The invention of the present disclosure may be a non-transitory computer readable medium having instructions stored thereon that, when executed by a processing device, cause the processing device to carry out an operation of clinical result aggregation, the operation comprising receiving one or more selected clinical trials, wherein the one or more selected clinical trials match a specification; and obtaining clinical trial results for the one or more selected clinical trials from at least one external data source. In an embodiment, the operation further comprises interpreting, via a machine learning model, the obtained clinical trial results; importing the obtained clinical trial results as structured data; and matching, based on a similarity analysis, via a processor, clinical trial endpoints identified in the obtained clinical trial results to corresponding normalized endpoint options. In a further embodiment, the operation further comprises aggregating, based on the matched corresponding normalized endpoint options, the obtained clinical trial results to determine aggregated results; and providing the aggregated results.

In an aspect of this disclosure, a computer program product embodied in a non-transitory computer readable medium comprises computer instructions for receiving one or more selected clinical trials, wherein the one or more selected clinical trials match a specification; obtaining clinical trial results for the one or more selected clinical trials from at least one external data source; interpreting, via a machine learning model, the obtained clinical trial results; importing the obtained clinical trial results as structured data; matching, based on a similarity analysis, via a processor, clinical trial endpoints identified in the obtained clinical trial results to corresponding normalized endpoint options; aggregating, based on the matched corresponding normalized endpoint options, the obtained clinical trial results to determine aggregated results; and providing the aggregated results.

Finally, other implementations of the disclosure will be apparent to those skilled in the art from consideration of the specification and practice of the disclosure disclosed herein. It is intended that the specification and examples be considered as exemplary only, with a true scope and spirit of the disclosure being indicated by the following claims.

Various elements, which are described herein in the context of one or more embodiments, may be provided separately or in any suitable subcombination. Further, the processes described herein are not limited to the specific embodiments described. For example, the processes described herein are not limited to the specific processing order described herein and, rather, process blocks may be re-ordered, combined, removed, or performed in parallel or in serial, as necessary, to achieve the results set forth herein.

It will be further understood that various changes in the details, materials, and arrangements of the parts that have been described and illustrated herein may be made by those skilled in the art without departing from the scope of the following claims.

All references, patents and patent applications and publications that are cited or referred to in this application are incorporated in their entirety herein by reference. Finally, other implementations of the disclosure will be apparent to those skilled in the art from consideration of the specification and practice of the disclosure disclosed herein. It is intended that the specification and examples be considered as exemplary only, with a true scope and spirit of the disclosure being indicated by the following claims.

What is claimed is:

1. A computer-implemented method, comprising the steps of:
receiving a one or more selected clinical trials,
wherein the one or more selected clinical trials match a specification;
obtaining a set of clinical trial results for the one or more selected clinical trials from at least a one external data source;
interpreting, via a machine learning model, the set of clinical trial results;
importing the set of clinical trial results in a structured data format;
matching, based on a similarity analysis, via a processor, a set of clinical trial endpoints identified in the set of clinical trial results to a set of corresponding normalized endpoint options;
aggregating, based on the set of corresponding normalized endpoint options, the set of clinical trial results to determine a set of aggregated results; and
providing the set of aggregated results.

2. A system, comprising:
a server comprising a at least one server processor, a at least one server database, a at least one server memory comprising a set of computer-executable server instructions which, when executed by the at least one server processor, cause the server to:
receive a one or more selected clinical trials,
wherein the one or more selected clinical trials match a specification;
obtain a set of clinical trial results for the one or more selected clinical trials from a at least one external data source;
interpret, via a machine learning model, the set of clinical trial results;
import the set of clinical trial results in a structured data format;
match, based on a similarity analysis, via a processor, a set of clinical trial endpoints identified in the set of clinical trial results to a set of corresponding normalized endpoint options;
aggregate, based on the set of corresponding normalized endpoint options, the set of clinical trial results to determine a set of aggregated results; and
provide the set of aggregated results; and
a client device comprising at least one device processor, at least one display, at least one device memory comprising computer-executable device instructions which, when executed by the at least one device processor, cause the client device to:
receive, from the client device, the specification via a graphical user interface.

3. The system of claim 2, wherein the one or more selected clinical trials are provided from a list comprising a one or more clinical trials matching the specification.

4. The system of claim 2, wherein the list identifies which of the one or more clinical trials matching the specification include a set of clinical result data obtainable from the at least one external data source.

5. The system of claim 2, wherein the specification comprises a disease category.

6. The system of claim 2, wherein the specification further comprises a specific disease within the disease category.

7. The system of claim 2, wherein the specification comprises a clinical trial phase category.

8. The system of claim 2, further comprising a client device comprising at least one device processor, at least one display, at least one device memory comprising a set of computer-executable device instructions which, when executed by the at least one device processor, cause the client device to receive, from a user, the specification via a graphical user interface.

9. The system of claim 2, wherein the at least one external data source comprises an online database of clinical trial data maintained by an at least one government entity responsible for regulating clinical trials, international agencies, university network organizations, organizations of medical associations, or foundations based on an association of pharmaceutical manufacturers.

10. The system of claim 2, wherein the machine learning model includes a named entity recognition (NER) model.

11. The system of claim 2, wherein the NER model utilizes a recurrent neural network (RNN) architecture.

12. The system of claim 2, wherein the set of computer-executable server instructions which, when executed by the at least one server processor, cause the server to interpret, via the machine learning model, the set of clinical trial results further cause the server to automatically extract specified text from unstructured text of the set of clinical trial results.

13. The system of claim 2, wherein the structured data format comprises a collection of fields corresponding to categories of syntactic units extracted from the set of clinical trial results.

14. The system of claim 2, wherein the similarity analysis comprises at least a computation of a word distance metric between the set of clinical trial endpoints identified in the set of clinical trial results and the set of corresponding normalized endpoint options.

15. The system of claim 2, wherein the set of computer-executable server instructions which, when executed by the at least one server processor, further cause the server to:
generate a one or more confirmation selection tools, the one or more confirmation selection tools corresponding to the set of corresponding normalized endpoint options; and
receive, from a user, actuation of one or more of the one or more confirmation selection tools.

16. The system of claim 2, wherein the set of aggregated results are ordered based on a match score of each result of the set of aggregated results.

17. The system of claim 2, wherein the set of aggregated results comprise a tabular structure comprising a one or more columns and a one or more rows, and wherein the one or more columns represent different clinical trials and the one or more rows represent different clinical trial properties.

18. The system of claim 2, wherein the set of computer-executable server instructions which, when executed by the at least one server processor, further cause the server to execute one or more selected from the group comprised of: filter, machine translate, and standardize terminology of the one or more selected clinical trials before obtaining the set of clinical trial results from the at least the one external data source.

19. The system of claim 2, wherein the machine learning model has been trained on a set of training datasets comprising a constrained set of collections of text with prescribed clinical endpoint categories to which the set of clinical trial endpoints identified in the set of clinical trial results belong.

20. A non-transitory computer readable medium having a set of instructions stored thereon that, when executed by a processing device, cause the processing device to carry out an operation of clinical result aggregation, the operation comprising:
receiving a one or more selected clinical trials,
wherein the one or more selected clinical trials match a specification;
obtaining a set of clinical trial results for the one or more selected clinical trials from a at least one external data source;
interpreting, via a machine learning model, the set of clinical trial results;
importing the set of clinical trial results in a structured data format;
matching, based on a similarity analysis, via a processor, a set of clinical trial endpoints identified in the set of clinical trial results to a set of corresponding normalized endpoint options;
aggregating, based on the set of corresponding normalized endpoint options, the set of clinical trial results to determine a set of aggregated results; and
providing the set of aggregated results.

21. A computer-implemented method, comprising the steps of:
receiving a one or more selected clinical trials;
obtaining a set of clinical trial results for the one or more selected clinical trials;
interpreting, via a machine learning model, the set of clinical trial results;
matching, based on a similarity analysis, via a processor, a set of clinical trial endpoints identified in the set of clinical trial results to a set of corresponding normalized endpoint options; and
aggregating, based on the set of corresponding normalized endpoint options, the set of clinical trial results to determine a set of aggregated results.
